(19) United States
(12) Patent Application Publication
(10) Pub. No.: US 2024/0280591 A1
(43) Pub. Date: Aug. 22, 2024

(54) BLOOD-BASED ASSAY FOR DETECTING TAUOPATHY OR AMYLOIDOGENIC DISEASE

(71) Applicant: Janssen Pharmaceutica NV, Beerse (BE)

(72) Inventors: Hartmuth Christian KOLB, San Diego, CA (US); Gallen TRIANA-BALTZER, San Diego, CA (US); Ziad Serhal SAAD, Encinitas, CA (US)

(73) Assignee: Janssen Pharmaceutica NV, Beerse (BE)

(21) Appl. No.: 18/627,010

(22) Filed: Apr. 4, 2024

Related U.S. Application Data
(63) Continuation of application No. 17/305,764, filed on Jul. 14, 2021, now abandoned.
(60) Provisional application No. 62/705,759, filed on Jul. 14, 2020.
(60) Provisional application No. 63/200,399, filed on Mar. 4, 2021.

(51) Int. Cl. G01N 33/68

(52) U.S. Cl. CPC G01N 33/6896; G01N 2800/2821; G01N 2800/50

(57) ABSTRACT

A method for detecting p217+tau in blood-based samples from a subject with high sensitivity, accuracy, and precision. The assay comprises contacting a sample with a capture antibody directed against a p217+tau epitope to bind the capture antibody to p217+tau peptides in plasma to form antibody-peptide complexes, and separately contacting the antibody-peptide complexes with a detection antibody to bind the detection antibody to the antibody-peptide complexes. The amount of p217+tau is determined by detecting the detection antibody. The amount of p217+tau detected is used to determine whether the subject has tauopathy or is at risk of developing tauopathy, or whether the subject has amyloidogenic disease or is at risk of developing amyloidogenic disease when the amount of p217+tau peptides is above a predetermined threshold value. The method has improved sensitivity such that the predetermined threshold value is above a Lower Limit of Quantification and/or Lower Limit of Detection of the assay.

### CROSS-REFERENCE TO RELATED APPLICATIONS

The present application is a continuation of U.S. application Ser. No. 17/305,764 filed Jul. 14, 2021, which claims priority to U.S. Provisional Application Ser. No. 62/705,759 filed Jul. 14, 2020 and U.S. Provisional Application Ser. No. 63/200,399 filed Mar. 4, 2021, the entire contents of which are hereby incorporated by reference herein.

### SEQUENCE LISTING

The present application contains a Sequence Listing which has been submitted electronically in ASCII format and is hereby incorporated by reference in its entirety. Said ASCII copy, created on Jun. 30, 2021, is named JAB7064_SL.txt and is 19,357 bytes in size.

### FIELD OF INVENTION

The present application relates to methods for detecting tauopathy and/or amyloidogenic disease. In particular, the present application invention relates to methods of measuring an amount of singly- or multiply-phosphorylated p217+tau protein species in a blood-based sample and uses thereof.

### BACKGROUND OF INVENTION

Alzheimer's Disease (AD) is a degenerative brain disorder characterized clinically by progressive loss of memory, cognition, reasoning, judgment, and emotional stability that gradually leads to profound mental deterioration and ultimately death. AD is a very common cause of progressive mental failure (dementia) in aged humans. More than 5 million people in the United States are living with AD, and the number is growing with an aging population. Indeed, 10% of people over age 65 have AD, and it is the 5th leading cause of death in this population. Overall AD is the 6th leading cause of death in the United States (1 in 3 seniors die with AD or another dementia), and it is estimated to cost the US $305 billion in 2020. AD has also been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

Neurofibrillary tangles are primarily composed of aggregates of hyper-phosphorylated tau protein. The main physiological function of tau is microtubule polymerization and stabilization. The binding of tau to microtubules takes place by ionic interactions between positive charges in the microtubule binding region of tau and negative charges on the microtubule lattice (Butner and Kirschner, J Cell Biol. 115(3):717-30, 1991). Tau protein contains 85 possible phosphorylation sites, and phosphorylation at many of these sites interferes with the primary function of tau. Tau that is bound to the axonal microtubule lattice is in a hypo-phosphorylation state, while aggregated tau in AD is hyper-phosphorylated, providing unique epitopes that are distinct from the physiologically active pool of tau (Iqbal et al., Curr Alzheimer Res. 7(8): 656-664, 2010).

The progression of tauopathy in an AD brain follows distinct spreading patterns. A tauopathy transmission and spreading hypothesis has been described based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009). It is believed that tauopathy can spread in a prion-like fashion from one brain region to the next. This spreading process would involve an externalization of tau seeds that can be taken up by nearby neurons and induce further tauopathy.

Numerous biochemical changes can be detected up to 20 years before onset of symptoms. The National Institute on Aging and Alzheimer's Association (NIA-AA) Research Framework provides a mechanism for the diagnosis of Alzheimer's Disease (AD) based on measurements that relate to the underlying pathologic processes, β-amyloid (A), pathologic tau (T) and neurodegeneration (N). Positron emission tomography using tau-specific radiotracers (Tau PET) has been used for measuring tau neurofibrillary tangles (NFTs) pathology in patients. However, Tau PET is a costly and cumbersome procedure and availability of tau-specific radiotracers may be limited.

Fragments of tau protein in the neurofibrillary tangles move to the cerebrospinal fluid (CSF) where they can be harvested and measured by sensitive assays. The presence of neurological disease can thus be detected using assays that recognize tau protein-derived fragments in CSF. However, retrieval of CSF requires patients to undergo invasive lumbar puncture procedures involving physicians inserting a needle into the spinal canal to collect samples of CSF for use in assays. Such procedures are uncomfortable and burdensome, and therefore, are not desirable for repeating frequently and not suitable for regular monitoring of disease states in patients.

### BRIEF SUMMARY OF THE INVENTION

One exemplary embodiment of the present application is directed to an assay method of detecting p217+tau peptides in a subject. The method comprises contacting a plasma sample with a capture antibody directed against a p217+tau epitope to bind the capture antibody to p217+tau peptides in the plasma to form antibody-peptide complexes, and washing the antibody-peptide complexes. The method then proceeds to contact the antibody-peptide complexes with a detection antibody to bind the detection antibody to the antibody-peptide complexes. The method then detects the detection antibody to determine an amount of the p217+tau peptides in the plasma sample.

A method of detecting tauopathy in a subject is also provided. The method comprises obtaining a plasma sample from the subject and detecting an amount of the p217+tau peptides present in the plasma sample using an assay. The assay uses a capture antibody directed against a p217+tau epitope to bind the capture antibody to p217+tau peptides in the plasma to form antibody-peptide complexes and a detection antibody to bind the detection antibody to the antibody-peptide complexes. The method further includes a step for determining the subject as having tauopathy or is at risk of developing tauopathy when the amount of p217+tau peptides is above a predetermined threshold value. The predetermined threshold value being above a Lower Limit of Quantification (LLOQ) of the assay.

A method of detecting amyloidogenic disease in a subject is also provided. The method comprises obtaining a plasma sample from the subject and detecting an amount of the p217+tau peptides present in the plasma sample using an assay. The assay uses a capture antibody directed against a p217+tau epitope to bind the capture antibody to p217+tau peptides in the plasma to form antibody-peptide complexes and a detection antibody to bind the detection antibody to the antibody-peptide complexes. The method further includes a step for determining the subject as having amyloidogenic disease or is at risk of developing amyloidogenic disease when the amount of p217+tau peptides is above a predetermined threshold value. The predetermined threshold value being above a Lower Limit of Quantification (LLOQ) of the assay.

In another aspect of the present application, a method for detecting or predicting tauopathy in a subject is provided. The method comprises detecting an amount of p217+tau peptides in a plasma sample by contacting the plasma sample with a capture antibody directed against a p217+tau epitope to bind the capture antibody to p217+tau peptides in the plasma to form antibody-peptide complexes, and separately contacting the antibody-peptide complexes with a detection antibody to bind the detection antibody to the antibody-peptide complexes, and generating tau data corresponding to the amount of p217+tau peptides detected. The method also comprises obtaining biomarker data corresponding to at least one biomarker detected from the patient, wherein the biomarker is selected from a group comprising NFL, adiponectin and leptin. The method further comprises comparing the tau data and the further biomarker data to a set of reference data using a machine learning module to determine or predict whether the subject has tauopathy or is at risk of developing tauopathy.

These and other aspects of the invention will become apparent to those skilled in the art after a reading of the following detailed description of the invention, including the figures and appended claims.

### BRIEF DESCRIPTION OF THE DRAWINGS

FIG. 1a shows data for p217+tau detected from plasma at 1:4 dilution obtained according to a previously disclosed assay.

FIG. 1b shows data for p217+tau detected from plasma at 1:16 dilution obtained according to the previously disclosed assay.

FIG. 1c shows data for p217+tau detected from semi-denatured samples of plasma obtained according to the previously disclosed assay.

FIG. 1d shows data for p217+tau detected from immunoprecipitated samples of plasma obtained according to the previously disclosed assay.

FIG. 2a shows data demonstrating correlation between p217+tau measurements from CSF obtained according to the previously disclosed assay and p217+tau measurements from CSF obtained according to an exemplary assay of the present application.

FIG. 2b shows data comparing p217+tau levels detected in serum obtained according to the previously disclosed assay and p217+tau levels detected in serum obtained according to an exemplary assay of the present application.

FIG. 3a shows data demonstrating correlation between p217+tau measurements from CSF obtained using two different detection antibodies according to exemplary embodiments of the present application.

FIG. 3b shows data demonstrating correlation between p217+tau measurements from serum obtained using two different detection antibodies according to exemplary embodiments of the present application.

FIG. 3c shows data demonstrating correlation between p217+tau measurements from plasma obtained using two different detection antibodies according to exemplary embodiments of the present application.

FIG. 4a shows data comparing effect of different sample diluents on bead clumping in exemplary embodiments of the present application.

FIG. 4b shows data comparing effect of different sample diluents on p217+tau levels detected by exemplary embodiments of the present application.

FIG. 5a shows data for p217+tau detected from serum obtained according to an exemplary embodiment of the present application.

FIG. 5b shows data for p217+tau detected from plasma obtained according to an exemplary embodiment of the present application.

FIG. 5c shows data demonstrating correlation between p217+tau detected from serum as shown in FIG. 5b and p217+tau detected from plasma as shown in FIG. 5c.

FIG. 5d shows data demonstrating correlation between p217+tau detected from plasma as shown in FIG. 5c and p217+tau detected from serum as shown in FIG. 5b.

FIG. 6a shows representative calibration curves generated using a calibrant peptide for an exemplary embodiment of an assay of the present application.

FIG. 6b shows data demonstrating dilution linearity of an exemplary assay of the present application in serum and plasma.

FIG. 7a shows data demonstrating intra-test precision of an exemplary assay of the present application in plasma.

FIG. 7b shows data demonstrating inter-test precision of an exemplary assay of the present application in plasma.

FIG. 7c shows further data demonstrating intra-test precision of an exemplary assay of the present application in plasma.

FIG. 8a shows data demonstrating correlation of p217+tau detected in CSF to p217+tau detected in plasma using an exemplary assay of the present application in AD subjects.

FIG. 8b shows further data demonstrating correlation of p217+tau detected in CSF to p217+tau detected in plasma using an exemplary assay of the present application in AD subjects.

FIG. 9a shows further data demonstrating correlation of p217+tau detected in CSF to p217+tau detected in plasma using an exemplary assay of the present application in a validation cohort.

FIG. 9b shows a Receiver-Operating Characteristic (ROC) curve of the data of FIG. 7b indicating the sensitivity of plasma measurement obtained according to an exemplary assay of the present application in differentiating brain pathologies of tauopathy.

FIG. 10a shows data demonstrating correlation of p217+tau detected in CSF to tau accumulation in brain tissue detected by Positron emission tomography (PET) imaging.

FIG. 10b shows a ROC curve of the data of FIG. 7b indicating the sensitivity of p217+tau measurements in CSF in differentiating brain pathologies of tauopathy.

FIG. 11a shows further data demonstrating correlation of p217+tau detected in CSF to p217+tau detected in plasma using an exemplary assay of the present application in a validation cohort.

FIG. 11b shows a subset of the data of FIG. 11a for amyloid positive patients having a CSF Aβ42/40 ratio of <0.089.

FIG. 11c shows a subset of the data of FIG. 11a for amyloid negative patients having a CSF Aβ42/40 ratio of >0.089.

FIG. 12a shows data demonstrating correlation of p181tau detected in CSF to p217+tau detected in plasma using an exemplary assay of the present application.

FIG. 12b shows a ROC curve of the data of FIG. 12c indicating the sensitivity of p217+tau measurements in plasma in differentiating CSF p217+tau levels.

FIG. 12c shows the data of FIG. 11a with threshold values for plasma p217+tau and CSF p217+tau for differentiating patients who have tauopathy or are at risk of developing tauopathy from those patients that are not at risk of developing tauopathy.

FIG. 12d shows a ROC curve of the data of FIG. 12e indicating the sensitivity of p217+tau measurements in plasma in differentiating CSF p217+tau levels.

FIG. 12e shows a subset of the data of FIG. 12c for cognitively normal subjects.

FIG. 12f shows a ROC curve of the data of FIG. 12g indicating the sensitivity of p217+tau measurements in plasma in differentiating CSF p217+tau levels.

FIG. 12g shows a subset of the data of FIG. 12c for mild-moderate dementia subjects.

FIG. 13a shows a ROC curve of the data of FIG. 13b indicating the sensitivity of p217+tau measurements in plasma in differentiating Aβ42/40 ratios in CSF.

FIG. 13b shows data demonstrating correlation of Aβ42/40 ratio in CSF to p217+tau detected in plasma using an exemplary assay of the present application.

FIG. 13c shows a ROC curve of the data of FIG. 13d indicating the sensitivity of p217+tau measurements in plasma in differentiating Aβ42/40 ratios in CSF.

FIG. 13d shows a subset of the data of FIG. 13a for cognitively normal subjects.

FIG. 13e shows a ROC curve of the data of FIG. 13f indicating the sensitivity of p217+tau measurements in plasma in differentiating ratios Aβ42/40 ratios in CSF.

FIG. 13f shows a subset of the data of FIG. 13a for mild-moderate dementia subjects.

FIG. 14a shows data demonstrating correlation of p217+tau detected in CSF to p217+tau detected in crude plasma using an exemplary assay of the present application.

FIG. 14b shows data demonstrating correlation of p217+tau detected in CSF to p217+tau detected in chemically extracted plasma using an exemplary assay of the present application.

FIG. 14c shows data demonstrating correlation of p217+tau detected in CSF to p217+tau detected in semi-denatured plasma using an exemplary assay of the present application.

FIG. 15a shows data for p217+tau detected from semi-denatured samples of plasma obtained according to an exemplary embodiment of an assay of the present application.

FIG. 15b shows representative calibration curves generated using a calibrant peptide for another exemplary embodiment of an assay of the present application, in which samples are semi-denatured prior to measurement.

FIG. 15c shows data demonstrating intra-test precision the exemplary assay of FIG. 9b, in which samples are semi-denatured prior to measurement.

FIG. 16a shows a ROC curve for a machine learning method for differentiating brain pathologies of tauopathy using serum p217+tau levels are a biomarker feature according to an exemplary embodiment of the present application.

FIG. 16b shows a ROC curve for a machine learning method for differentiating brain pathologies of tauopathy using serum p217+tau levels and data for neurofilament light chain (NFL) as biomarker features according to an exemplary embodiment of the present application.

FIG. 16c shows a ROC curve for a machine learning method for differentiating brain pathologies of tauopathy using serum p217+tau levels and data for NFL and adiponectin as biomarker features according to an exemplary embodiment of the present application.

FIG. 16d shows a ROC curve for a machine learning method for differentiating brain pathologies of tauopathy using serum p217+tau levels and data for NFL, adiponectin and leptin as biomarker features according to an exemplary embodiment of the present application.

FIG. 16e shows a ROC curve for a machine learning method for differentiating brain pathologies of tauopathy using data for NFL, adiponectin and leptin as biomarker features according to an exemplary embodiment of the present application.

### DETAILED DESCRIPTION OF THE INVENTION

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It is noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “antibody” or “immunoglobulin” refers to a specific protein capable of binding an antigen or portion thereof. These terms are used herein in a broad sense and includes immunoglobulin or antibody molecules including polyclonal antibodies, monoclonal antibodies (including murine, human, human-adapted, humanized and chimeric monoclonal antibodies) and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Accordingly, the antibodies of the present application can be of any of the five major classes or corresponding sub-classes. Preferably, the antibodies of the present application are IgG1, IgG2, IgG3 or IgG4. Antibody light chains of any vertebrate species can be assigned to one of two clearly distinct types, namely kappa and lambda, based on the amino acid sequences of their constant domains. Accordingly, the antibodies of the present application can contain a kappa or lambda light chain constant domain. According to particular embodiments, the antibodies of the present application include heavy and/or light chain constant regions from mouse antibodies or human antibodies.

In addition to the heavy and light constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J Exp Med. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVRs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database (http:_//www_imgt_org) provides a standardized numbering and definition of these regions. The correspondence between CDRs, HVRs and IMGT delineations is described in Lefranc et al., 2003, Id.;
(iv) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a bispecific or multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the constant region of the heavy chain. According to other particular embodiments, the antigen-binding fragment comprises Fab and F(ab′).

As used herein, the term “epitope” refers to a site on an antigen to which an immunoglobulin, antibody, or antigen-binding fragment thereof, specifically binds. Epitopes can be formed both from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as 0N3R, 1N3R, 2N3R, 0N4R, 1N4R and 2N4R. As used herein, the term “control tau” refers to the tau isoform of SEQ ID NO: 1 that is devoid of phosphorylation and other post-translational modifications. As used herein, the term “tau” includes proteins comprising mutations, e.g., point mutations, fragments, insertions, deletions and splice variants of full-length wild type tau. The term “tau” also encompasses post-translational modifications of the tau amino acid sequence. Post-translational modifications include, but are not limited to, phosphorylation.

Unless otherwise indicated, as used herein, the numbering of the amino acid in a tau protein or fragment thereof is with reference to the amino acid sequence set forth in SEQ ID NO: 1.

As used herein, the term “p217+tau peptides,” “p217+tau,” or “p217+tau protein” means a human tau protein or tau fragment that is phosphorylated at residue 217 (pT217) of tau protein, and may or may not be further phosphorylated at additional residues, such as, for example, residue 212 (pT212) of tau protein, wherein the numbering of the positions is according to the numbering in SEQ ID NO: 1.

As used herein, the term “p217+tau epitope” refers to a tau epitope containing at least one of phosphorylated T217 and phosphorylated T212, wherein the numbering of the positions is according to the numbering in SEQ ID NO: 1. Examples of p217+tau epitope include, e.g., a pT3 epitope. As used herein, the term “pT3 epitope” refers to an epitope containing amino acids 210-220 of human tau protein that is phosphorylated at residue 217, and may or may not be further phosphorylated at additional residues, such as, for example, residue 212, wherein the numbering of the positions is according to the numbering in SEQ ID NO: 1.

As used herein, each of the terms “long p217+tau peptides,” “long p217+tau,” “long form of p217+tau peptides,” or “long p217+tau peptides fragment” has the same meaning, referring to a p217+tau peptides that comprises the p217+tau epitope and an epitope comprising amino acid residues 7 to 20 of tau protein. The “long p217+tau peptides” according to embodiments of the present application can have different lengths. For example, the amino-terminus of a “long p217+tau peptides fragment” can be the amino acid residue 1, 2, 3, 4, 5, 6, or 7 of tau protein. In one example, the “long p217+tau peptides,” may comprise amino acid residues 7 to 220 of p217+tau protein.

As used herein, each of the terms “short p217+tau peptides,” “short p217+tau,” “short form of p217+tau peptides,” or “short p217+tau peptides fragment” has the same meaning, referring to a p217+tau peptides that comprises the p217+tau epitope and an epitope comprising amino acid residues 119 to 126 of tau protein, but does not contain an epitope comprising amino acid residues 7 to 20 of tau protein. The “short p217+tau peptides” according to embodiments of the present application can have different lengths. For example, the amino-terminus of a “short p217+tau peptides” can be any of the amino acid residues that are between the epitope comprising amino acid residues 7 to 20 of tau protein and the epitope comprising amino acid residues 119 to 126 of tau protein. In one example, the “short p217+tau peptides” may comprise amino acid residues 119-220 of p217+tau protein.

As used herein, each of the terms “long tau peptide,” “long tau,” “long form of tau peptide,” or “long tau peptide fragment” has the same meaning, referring to a tau peptide that comprises the tau epitope recognized by a phosphorylation-independent capture antibody and an epitope comprising amino acid residues 7 to 20 of tau protein. The “long tau peptide fragments” according to embodiments of the present application can have different lengths. For example, the amino-terminus of a “long tau peptide fragment” can be the amino acid residue 1, 2, 3, 4, 5, 6, or 7 of tau protein.

As used herein, each of the terms “short tau peptide,” “short tau,” “short form of tau peptide,” or “short tau peptide fragment” has the same meaning, referring to a tau peptide that comprises the tau epitope recognized by a phosphorylation-independent capture antibody and an epitope comprising amino acid residues 119 to 126 of tau protein, but does not contain an epitope comprising amino acid residues 7 to 20 of tau protein. The “short tau peptide fragments” according to embodiments of the present application can have different lengths. For example, the amino-terminus of a “short tau peptide” can be any of the amino acid residues that are between the epitope comprising amino acid residues 7 to 20 of tau protein and the epitope comprising amino acid residues 119 to 126 of tau protein.

As used herein, the term “capture antibody” refers to an antibody that binds to an antigen of interest and is directly or indirectly linked to a solid support. Examples of solid supports include, but are not limited to, microparticles or beads, such as a magnetic beads or paramagnetic beads. Examples of capture antibodies include, but are not limited to, a monoclonal antibody that binds to a p217+tau epitope.

According to embodiments of the present application, the capture antibody can be a monoclonal antibody comprising immunoglobulin heavy chain HCDR1, HCDR2 and HCDR3 having the polypeptide sequences of SEQ ID NOs: 23, 24 and 25, respectively, and immunoglobulin light chain LCDR1, LCDR2 and LCDR3 having the polypeptide sequences of SEQ ID NOs: 26, 27 and 28. In a particular embodiment, the capture antibody is pT3. As used herein, the term “pT3” refers to an antibody that binds to p217+tau peptides and has a heavy chain variable region amino acid sequence of SEQ ID NO: 19 and a light chain variable region amino acid sequence of SEQ ID NO: 20. In one embodiment, the pT3 monoclonal antibody is expressed by a mouse-hybridoma. In another embodiment, the capture antibody is a humanized antibody having a heavy chain variable region amino acid sequence of SEQ ID NO: 21 and a light chain variable region amino acid sequence of SEQ ID NO: 22.

According to other embodiments of the present application, the capture antibody can be a monoclonal antibody that binds to an epitope between amino acids 150 and 250 of tau protein, preferably amino acids 211-221 or amino acids 159-163 of human tau protein, in a phosphorylation-independent manner, and the numbering of the positions is according to the numbering in SEQ ID NO: 1. In a particular embodiment, the capture antibody is hT7. As used herein, the term “hT7” refers to a publicly available monoclonal antibody that binds to an epitope comprising amino acids 159-163 of human tau protein, wherein the numbering of the positions is according to the numbering in SEQ ID NO: 1. A hT7 monoclonal antibody is commercially available, e.g., from ThermoFisher (e.g., Catalog #: MN1000).

As used herein, the term “detection antibody” refers to an antibody that binds to an antigen of interest and has a detectable label or is linked to a secondary detection system. Examples of detectable labels include, but are not limited to, various enzymes, prosthetic groups, fluorescent materials, luminescent materials, bioluminescent materials, and radioactive materials. Examples of detection antibodies include, but are not limited to, a monoclonal antibody that binds to tau protein, preferably an epitope comprising amino acids 7-20 or 116-127 of human tau protein, wherein the numbering of the positions is according to the numbering in SEQ ID NO: 1. When a monoclonal antibody that binds to a tau protein at an epitope comprising amino acids 7-20 is used as a detection antibody for captured p217+tau peptides, long tau fragments are detected. When a monoclonal antibody that binds to a tau protein at an epitope comprising amino acids 116-127 is used as a detection antibody for captured p217+tau peptides, both short and long tau fragments are detected.

According to embodiments of the present application, the detection antibody can be a monoclonal antibody comprising immunoglobulin heavy chain HCDR1, HCDR2 and HCDR3 having the polypeptide sequences of SEQ ID NOs: 10, 11, and 12, respectively, and immunoglobulin light chain LCDR1, LCDR2 and LCDR3 having the polypeptide sequences of SEQ ID NOs: 13, 14 and 15. In a particular embodiment, the detection antibody is hT43. As used herein, the term “hT43” refers to a monoclonal antibody that binds to an epitope comprising amino acids 7-20 of human tau protein, wherein the numbering of the positions is according to the numbering in SEQ ID NO: 1, and the antibody has a heavy chain variable region amino acid sequence of SEQ ID NO: 16 and a light chain variable region amino acid sequence of SEQ ID NO: 17.

In another embodiment, the detection antibody can be a monoclonal antibody comprising immunoglobulin heavy chain HCDR1, HCDR2 and HCDR3 having the polypeptide sequences of SEQ ID NOs: 2, 3 and 4, respectively, and immunoglobulin light chain LCDR1, LCDR2 and LCDR3 having the polypeptide sequences of SEQ ID NOs: 5, 6 and 7. In another particular embodiment, the detection antibody is pT82. As used herein, the term “pT82” refers to a monoclonal antibody that binds to an epitope comprising amino acids 119-126, preferably 117-127, of human tau protein, wherein the numbering of the positions is according to the numbering in SEQ ID NO: 1, and the antibody has a heavy chain variable region amino acid sequence of SEQ ID NO: 8 and a light chain variable region amino acid sequence of SEQ ID NO: 9.

As used herein, the term “pT3-based assay” refers to an assay wherein the pT3 antibody is used as the capture antibody. As used herein, the term “pT3xhT43” refers to an assay wherein the pT3 antibody is used as the capture antibody and the hT43 antibody is used as the detection antibody. As used herein, the term “pT3xpT82” refers to an assay wherein the pT3 antibody is used as the capture antibody and the pT82 antibody is used as the detection antibody.

As used herein, the term “hT7-based assay” refers to assays wherein the hT7 antibody is used as the capture antibody. As used herein, the term “hT7xpT82” refers to assays wherein the hT7 antibody is used as the capture antibody and the pT82 antibody is used as the detection antibody.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig, marmoset or mouse) or a primate (e.g., a monkey, chimpanzee, or human). In particular embodiments, the subject is a human.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

As used herein, the term “amyloidogenic disease” includes any disease associated with (or caused by) the formation or deposition of insoluble amyloid fibrils. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient. The terms “β-amyloid protein,” “β-amyloid peptide,” “β-amyloid,” “Aβ” and “Aβ peptide” are used interchangeably herein.

As used herein, the terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “diagnosis” means detecting a disease or disorder or determining the stage or degree of a disease or disorder, such as a tauopathy or an amyloidogenic disease. Usually, a diagnosis of a disease or disorder is based on the evaluation of one or more factors and/or symptoms that are indicative of the disease. A diagnosis can be made based on the presence, absence or amount of a factor which is indicative of presence or absence of the disease or condition, e.g., p217+tau. Each factor or symptom that is considered to be indicative for the diagnosis of a particular disease does not need be exclusively related to the particular disease, i.e., there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, e.g., an anti-p217+tau antibody therapy, or predicting the pattern of response to a drug therapy, e.g., an anti-p217+tau antibody therapy. The diagnostic methods may be used independently, or in combination with other diagnosing and/or staging methods known in the medical arts for a particular disease or disorder, e.g., Alzheimer's disease.

As used herein, the terms “increase” and “decrease” refer to differences in the quantity of a particular biomarker in a sample as compared to a control or reference level. For example, the quantity of particular peptide, may be present at an elevated amount or at a decreased amount in samples of patients with a disease compared to a reference level. In one embodiment, an “increase of a level” or “decrease of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, an “increase of a level” or “decrease of a level” may be a statistically significant difference between the level of the biomarker present in a sample as compared to a control. For example, a difference may be statistically significant if the measured level of the biomarker falls outside of about 1.0 standard deviation, about 1.5 standard deviations, about 2.0 standard deviations, or about 2.5 standard deviations of the mean of any control or reference group. The reference or control can be, for example, a sample from a healthy individual, or a sample taken from the same individual at an earlier time point, such as a time point prior to administration of a therapeutic or an earlier time point during a therapeutic regimen.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

An “isolated antibody that binds to a tau protein” or an “isolated anti-tau antibody”, as used herein, is intended to refer to an antibody that specifically binds tau protein and which is substantially free of other antibodies having different antigenic specificities (for instance, an isolated anti-tau detection antibody is substantially free of antibodies that specifically bind antigens other than tau). An isolated anti-tau detection antibody can, however, have cross-reactivity to other related antigens, for instance from other species (such as tau species homologs).

As used herein, the term “specifically binds” or “specific binding” refers to the ability of an anti-tau antibody of the present application to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The K_{D} is obtained from the ratio of Kd to Ka (i.e., Kd/Ka) and is expressed as a molar concentration (M). K_{D} values for antibodies can be determined using methods in the art in view of the present disclosure. For example, the K_{D} value of an anti-tau antibody can be determined by using surface plasmon resonance, such as by using a biosensor system, e.g., a Biacore® system, a Proteon instrument (BioRad), a KinExA instrument (Sapidyne), ELISA or competitive binding assays known to those skilled in the art. Typically, an anti-tau antibody binds to a predetermined target (i.e., tau) with a K_{D} that is at least ten fold less than its K_{D} for a nonspecific target as measured by surface plasmon resonance using, for example, a Proteon Instrument (BioRad). The anti-tau antibodies that specifically bind to tau can, however, have cross-reactivity to other related targets, for example, to the same predetermined target from other species (homologs), such as from mouse, rat, marmoset, dog or pig.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein the term “modulating, ameliorating, or treating” or “treatment” includes prophylaxis of a physical and/or mental condition or amelioration or elimination of the developed physical and/or mental condition once it has been established or alleviation of the characteristic symptoms of such condition.

As used herein, the term “accuracy” refers to the degree of closeness of a valued to the true value of an assay.

As used herein, the term “precision” refers to closeness of agreement among a series of measurements obtained from multiple samplings of the same homogenous sample of an assay.

The term “sensitivity” as used herein refers to the lowest analyte concentration in a sample that can be measured with acceptable accuracy and precision in an assay.

The present application provides assays and methods for detecting singly- or multiply-phosphorylated p217+tau peptides in blood-based samples, in particular, plasma. Collection of blood samples is fast and easy to perform and provides a reduced risk of infection or other complications as compared to lumbar puncture used for collection of CSF. Assays and methods of the present application measure p217+tau peptides in blood-based samples with sufficient sensitivity, precision and accuracy. Therefore, the present application provides an improved way for measuring and/or monitoring p217+tau levels in subjects as compared to CSF-based assays, by minimizing the burden of sample collection on subjects and thereby enabling more frequent assaying and monitoring of changes in p217+tau levels, which is particularly desired to monitor and evaluate response to a treatment. The sample used in assays and methods of the present application may be a blood, serum, or plasma sample. Preferably, the sample is a plasma sample. More preferably, the plasma sample has not been immunoprecipitated to concentrate the p217+tau peptides contained therein. In a particular embodiment, the sample is a crude plasma sample.

The assays and methods of the present application are directed to measurement of p217+tau peptides in blood-based samples by using a capture antibody which binds to p217+tau peptides in the sample. The capture antibody is preferably immobilized to a solid phase so that the capture antibody selectively binds to and immobilizes the p217+tau peptides present in the sample to the solid phase. In a separate step, the captured p217+tau peptides are contacted with an anti-tau detection antibody, which is labeled with a reporter element that allows detection of the captured p217+tau species. The assays and methods described herein can be used for various diagnostic purposes, e.g., for diagnosing AD, other tauopathies, other diseases characterized by deposits of Aβ, or other amyloidogenic diseases in a subject, monitoring the effectiveness of a treatment, identifying a subject suitable for an anti-p217+tau treatment, pre-screening subjects for PET imaging and/CSF assays for further detection of AD, other tauopathies, other diseases characterized by deposits of Aβ, or other amyloidogenic diseases, identification of subjects for enrollment in clinical trials relating to AD, other tauopathies, other diseases characterized by deposits of Aβ, or other amyloidogenic diseases, etc.

In one exemplary embodiment, the assays and methods of the present application include steps for contacting a blood-based sample with a capture antibody directed against a p217+tau epitope to bind the capture antibody to p217+tau peptides in the sample to create antibody-peptide complexes, and then washing the antibody-peptide complexes. The antibody-peptide complexes may be washed with any suitable solution that does not interfere with the assays, such as, for example, a buffer solution (e.g., Phosphate-buffered saline (PBS) solution). The washed antibody-peptide complexes may then be contacted with a detection antibody to bind the detection antibody to the antibody-peptide complexes. The detection antibody is then detected to determine an amount of p217+tau peptides in the sample.

In U.S. Pat. No. 10,591,492 by Kolb et al. (herein after “Kolb '492 patent”), which is incorporated by reference in its entirety herein, an assay for measuring p217+tau peptides in biologic samples is disclosed. The biologic samples can include cerebral spinal fluid (CSF), blood or brain homogenate. It was observed in the Kolb '492 patent that tau measurements in crude serum or plasma do not exhibit ideal diagnostic performance and may be plagued by sensitivity and matrix interference hurdles. As demonstrated below, measurements in crude serum showed that the assay described in the Kolb '492 patent could not provide sufficient sensitivity as most of the measurements were below the Lower Limit of Quantification (LLOQ) of its assay, which refers to the lowest amount of an analyte that can be quantitatively determined with acceptable precision and accuracy, and was only shown to be detectable after the samples were immunoprecipitated followed by heat denaturing of the elute. Example 3 provided below, demonstrates that the pT3xpT82 assay described in the Kolb '492 patent, which combines a sample with both a capture antibody and a detection antibody in a single step within the same mixture, lacks sensitivity and dilution linearity when used to measure p217+tau peptides in plasma.

In contrast, although levels of p217+tau peptides are significantly lower in blood-based samples as compared to CSF, the assays and methods of the present application can surprisingly measure p217+tau peptides from human serum and/or plasma samples with improved sensitivity without first concentrating the p217+tau peptides in the samples by immunoprecipitation prior to measurement. Immunoprecipitation is a cumbersome and imprecise process. Therefore, the present application provides improved assays and methods that improve sensitivity for measuring p217+tau peptides without the burdensome pre-processing of immunoprecipitating the samples prior to measurement. In particular, it has been surprisingly found that separate steps (1) for binding the capture antibody to the p217+tau peptides present in serum and/or plasma samples to form antibody-peptide complexes, and (2) for binding the antibody-peptide complexes to the detector antibody, can successfully reduce interference from other components (e.g., endogenously produced or exogenously administered interfering antibodies) in the sample, such that the assay is sufficiently sensitive for detecting p217+tau peptides in serum and/or plasma. In one embodiment, the sample may first be contacted with the capture antibody to bind the capture antibody to p217+tau peptides in the sample, and then washed to remove any unbound components that may interfere with the assay. After the wash, the captured p217+tau peptides are then contacted with the detection antibody to bind the detection antibody to the captured p217+tau peptides. Example 4 provided below further demonstrates that when the assay described in the Kolb '492 patent, which combines a sample with both a capture antibody and a detection antibody in a single step within the same mixture, is used to measure p217+tau peptides in serum, an artifact signal corresponding to interfering components is observed, which was not observed in CSF. However, this artifact signal is not present for p217+tau measurements obtained using an exemplary method comprising separate steps for binding the capture antibody and the detector antibody to the p217+tau peptides, as described in the present application.

In addition, it has also been surprisingly found that assays and methods of the present application is surprisingly more sensitive when measuring p217+tau peptides from human plasma samples as compared to human serum samples due to a surprisingly increased level of p217+tau detectable in plasma, as shown further below in Example 7. The assays and methods of the present application are able to measure p217+tau peptides and provide accurate and precise quantitative results for both healthy subjects and those subjects having or at risk of developing tauopathy, and more specifically, AD. The assays and methods of the present application are also able to measure p217+tau peptides and provide accurate and precise quantitative results for healthy subjects and those subjects having or at risk of developing amyloidogenic diseases, in particular in subjects having dementia (e.g., mild-moderate dementia). Specifically, the assays and methods are able to measure p217+tau peptides from plasma samples of both groups of subjects above the LLOQ of the assays of the present application, indicating an acceptable and reliable level of sensitivity. The LLOQ of the assays may be within 15%-25% of the assays' coefficient of variation (CV), within 15%-20% of the CV, or preferably within 20% of the CV. Furthermore, p217+tau measurements obtained according to the assays and methods of the present application from plasma samples of healthy subjects are numerically separable from those measurements from AD subjects. Therefore, the assays and methods of the present application provides accurate and precise measurements that can be used to identify healthy subjects from AD subjects.

In one embodiment, the assays and methods of the present application measure p217+tau from a plasma sample from a subject and subsequently determines that the subject has or is at risk of developing tauopathy and/or amyloidogenic disease when the amount of p217+tau measured from the plasma sample is above a predetermined threshold value. The predetermined threshold value may be any suitable threshold value for distinguishing those subjects who have or are at risk of developing tauopathy and/or amyloidogenic disease as compared to those subjects who are healthy and not at risk of developing tauopathy and/or amyloidogenic disease. The predetermined threshold value may be determined as a plasma p217+tau concentration for: differentiating those patients above a level of tau in the brain or regions of the brain as measured by PET imaging and those below; differentiating those patients above a level of tau (e.g., phosphorylated tau such as p181 or p217+tau) in CSF and those below; differentiating those patients above a level of β-amyloid (e.g., Aβ40 or Aβ42), such as in CSF or in plasma; differentiating those patients above a ratio of Aβ42 to Aβ40, such as in CSF or in plasma, and those below; and differentiating those patients that are cognitively normal and those patients that have dementia. Because of the surprisingly higher sensitivity of the assays and the methods of the present application in human plasma, the predetermined threshold value is above the LLOQ of the assay, thereby providing a sensitive, quantifiable threshold level for identify those subjects that have or are at risk of developing tauopathy and/or amyloidogenic separate from healthy subjects. In particular, the predetermined threshold value is above the LLOQ and/or Lower Limit of Detection (LLOD) of the assay, which is the lowest amount of an analyte that can reliably be detected. For example, the predetermined threshold value may be at least 3, 4, 5, 7 or 10 times the LLOQ of the assay and/or at least 10, 20, 30, 40, 50, 60, 70, 80, 90, 100, 110 or 120 times the LLOD of the assay.

Subjects identified as having or are at risk of developing tauopathy and/or amyloidogenic disease may be directed to obtain further clinical tests, such as, for example, CSF collection and/or PET imaging, to further assess brain pathologies of these subjects. In another embodiment, subjects identified as having or are at risk of developing tauopathy and/or amyloidogenic disease may be administered an active agent for treating cognitive decline or tauopathy and/or amyloidogenic disease, for example, AD. Active agents for treating tauopathy may include anti-tau antibodies, anti-p217+tau antibodies, small interfering RNA (siRNA) against human tau, siRNA against p217+tau, cholinesterase inhibitors, N-methyl D-aspartate (NMDA) antagonist, etc. Active agents for amyloidogenic disease may include anti-amyloid antibodies, beta secretase inhibitors, gamma secretase inhibitors, small interfering RNA (siRNA) against human β-amyloid, cholinesterase inhibitors, N-methyl D-aspartate (NMDA) antagonist, etc.

In some embodiments, the predetermined threshold value may correspond to a baseline value or a value that is significantly higher than the baseline value. As used herein, “significantly higher” refers to a higher value that is statistically significant, not due to chance alone, which has a p-value of 0.05 or less. “Significantly higher” can be at least about 1%, 2%, 5%, or 10% higher than that found in healthy volunteers, at a p-value of less than 0.05, 0.04, 0.03, 0.01, 0.005, 0.001, etc. The baseline value may correspond to a mean level in a population of healthy individuals. The baseline value may also correspond to a mean value of previous levels determined in the same subject.

In one embodiment, the capture antibody is a monoclonal antibody directed against a p217+tau epitope, and the detection antibody is a monoclonal antibody directed against an epitope comprising amino acid residues 7 to 20 of tau protein. In another embodiment, the capture antibody is a monoclonal antibody directed against a p217+tau epitope, and the detection antibody is a monoclonal antibody directed against an epitope comprising amino acid residues 119 to 126, preferably 116-127, of human tau protein. In one exemplary embodiment, the capture antibody is a monoclonal antibody comprising immunoglobulin heavy chain HCDR1, HCDR2 and HCDR3 having the polypeptide sequences of SEQ ID NOs: 23, 24, and 25, respectively, and immunoglobulin light chain LCDR1, LCDR2 and LCDR3 having the polypeptide sequences of SEQ ID NOs: 26, 27, and 28 and the detection antibody is a monoclonal antibody comprising immunoglobulin heavy chain HCDR1, HCDR2 and HCDR3 having the polypeptide sequences of SEQ ID NOs: 10, 11 and 12, respectively, and immunoglobulin light chain LCDR1, LCDR2 and LCDR3 having the polypeptide sequences of SEQ ID NOs: 13, 14 and 15. More particular, the capture antibody is pT3 and/or the detection antibody is hT43.

According to an embodiment of the present application, p217+tau peptides in a sample of interest are captured with a capture antibody directed against a p217+tau epitope. The captured p217+tau peptides, while all contain the p217+tau epitope, may have different length, which can be detected by detection antibodies binding to different epitopes. For example, a detection antibody directed against an epitope comprising amino acid residues 7 to 20 of tau protein can only detect captured p217+tau peptides or fragments thereof that still contain amino acid residues 7 to 20 of tau protein (“long p217+tau peptides”), while a detection antibody directed against an epitope comprising amino acid residues 119 to 126 of tau protein can detect not only the long p217+tau peptides, but also the short p217+tau peptides. The captured p217+tau peptides can be contacted with a detection antibody directed against an epitope comprising amino acid residues 7 to 20 or 116 to 127 of tau protein to thereby detect and measure the amount of the long p217+tau peptides or the p217+tau peptides (long and short p217+tau peptides) in the sample. An amount of short p217+tau peptides in a sample is calculated by subtracting the amount of long p217+tau peptides from the amount of p217+tau peptides.

In view of the surprisingly improved sensitivities observed for detecting p217+tau peptides using the assays and methods of the present application in plasma, it is believed that the improved sensitivities are equally applicable to detection of short p217+tau peptides and/or long p217+tau peptides described above. Therefore, in another embodiment of the present application, the assays and methods of the present application include measuring short p217+tau and/or long p217+tau peptides from serum and/or plasma samples. In particular, the assays and methods of the present application may measure short p217+tau and/or long p217+tau from human plasma with increased sensitivity. In a further embodiment, the assays and methods of the present application measure short p217+tau and/or long p217+tau from plasma sample from a subject and subsequently determines that the subject has or is at risk of developing tauopathy, wherein the amount of short p217+tau and/or long p217+tau peptides measured from the plasma sample are above predetermined threshold(s). The predetermined threshold(s) are above the LLOQs of the assays.

According to another embodiment of the present application, in addition to capturing and measuring the amount of p217+tau peptides in a sample, total tau peptides in the sample are captured with a phosphorylation-independent capture antibody, such as an antibody directed against an epitope between amino acids 150 and 250 of tau protein, preferably an epitope comprising amino acids 159-163 of tau protein. The captured total tau peptides can be contacted with a detection antibody directed against an epitope comprising amino acid residues 7 to 20 or 116 to 127 of tau protein to thereby detect and measure the amount of the total long tau peptides or the total tau peptides (long and short tau peptide fragments) in the sample. An amount of short total tau peptides in a sample is calculated by subtracting the amount of long total tau peptides from the amount of total tau peptides.

According to embodiments of the present application, a value related to p217+tau peptides in a sample, such as the amount of p217+tau peptides and the amount of long p217+tau peptides, optionally the amount of total tau peptides and the amount of total long tau fragments, in a sample, as well as information based on the measure amounts, such as the calculated short p217+tau peptides and short total tau peptides, or a ratio related to p217+tau peptides, such as a ratio of the amount of short tau peptide fragments to the amount of long tau peptide fragments, a ratio of the amount of short p217+tau peptides to the total amount of short tau fragments, a ratio of amount of long p217+tau peptides to the total amount of long tau fragments, etc., can be used for one or more diagnostic purposes. In one embodiment, it is determined that a subject is suffering from a tauopathy if a ratio related to p217+tau peptides, e.g., a ratio of the amount of short p217+tau peptides to the amount of long p217+tau peptides, is significantly higher than a corresponding baseline ratio and the amount of p217+tau, short p217+tau and/or long p217+tau measured is above the LLOQs of the assays.

In one embodiment, a method of the present application comprises (i) contacting a blood-based sample, preferably a plasma sample, with a capture antibody directed against an epitope comprising phosphorylated p217+tau to capture p217+tau peptides in the sample, (ii) separately contacting the captured p217+tau peptides with a detection antibody directed against an epitope comprising amino acid residues 7 to 20, preferably after washing the captured p217+tau peptide, to thereby measure the amount of long p217+tau peptides, and/or with a detection antibody directed against an epitope comprising amino acid residues 119 to 126 of tau protein, preferably after washing the captured p217+tau peptide, to thereby measure the amount of long and short p217+tau peptides in the sample, and (iii) determining whether or not the subject suffers from a tauopathy or is at risk of developing a tauopathy based on the amount of the p217+tau peptides or the ratio of the amount of short p217+tau peptides to the amount of long p217+tau peptides. Diagnosis can be performed by comparing the amount or concentration of p217+tau peptides in a sample from the subject to corresponding predetermined threshold levels. Diagnosis can also be performed by comparing the ratio of the amount of short p217+tau peptides to the amount of long p217+tau peptides in a sample from the subject to corresponding baseline ratios, wherein the amounts of short p217+tau peptides and long p217+tau peptides are above the LLOQs of their respective assays.

In another embodiment, a method of the present application comprises (i) contacting a blood-based sample, preferably a plasma sample, with a capture antibody directed against a p217+tau epitope to capture p217+tau peptides in the sample, and/or with a phosphorylation-independent capture antibody directed against a tau epitope between amino acids 150 and 250 of tau protein to capture total tau peptides in the sample, (ii) separately contacting the captured p217+tau peptides, or the captured total tau peptides, with a detection antibody directed against an epitope comprising amino acid residues 116 to 127 of tau protein, preferably after washing the captured p217+tau peptides or the captured total tau peptides, to thereby measure the amount of long and short p217+tau peptides, or the amount of total short tau peptides, in the sample, and (iii) determining whether or not the subject suffers from a tauopathy or is at risk of developing a tauopathy based on the ratio of the amount of short p217+tau peptides to the amount of total short tau peptides in the sample. Diagnosis can be performed by comparing the ratio of the amount of short p217+tau peptides to the amount of total short tau peptides comprising the same region of tau protein as that recognized by the pT3 antibody, i.e., amino acids 211-221 of tau, in a sample from the subject to corresponding baseline values, wherein the amount of short p217+tau peptides is above the LLOQ of the assay.

In another embodiment, a method of the present application comprises (i) contacting a blood-based sample, preferably a plasma sample, with a capture antibody directed against a p217+tau epitope to capture p217+tau peptides in the sample, (ii) separately contacting the captured p217+tau peptides with a detection antibody directed against an epitope comprising amino acid residues 7 to 20, preferably after washing the captured p217+tau peptides, to thereby measure the amount of long p217+tau peptides, and/or with a detection antibody directed against an epitope comprising amino acid residues 116 to 127 of tau protein, preferably after washing the captured p217+tau peptides, to thereby measure the amount of long and short p217+tau peptides in the sample, and (iii) determining the effectiveness of the treatment in the subject based on the amount of the p217+tau peptides or the ratio of the amount of short p217+tau peptides to the amount of long p217+tau peptides, wherein the amount(s) of the p217+tau peptides, short p217+tau peptides, and/or long p217+tau peptides measured are above the LLOQs of the respective assays.

In yet another embodiment, a method of the present application comprises (i) contacting a blood-based sample, preferably a plasma sample, with a capture antibody directed against a p217+tau epitope to capture p217+tau peptides in the sample, and/or with a phosphorylation-independent capture antibody directed against a tau epitope between amino acids 150 and 250 of tau protein to capture total tau peptides in the sample, (ii) separately contacting the captured p217+tau peptides, or the captured total tau peptides, with a detection antibody directed against an epitope comprising amino acid residues 116 to 127 of tau protein, preferably after washing the captured p217+tau peptides or the captured total tau peptides, to thereby measure the amount of long and short p217+tau peptides, or the amount of total short tau peptides, in the sample, and (iii) determining the effectiveness of the treatment in the subject based on the amount of the ratio of the amount of short p217+tau peptides to the amount of total short tau peptides in the biological sample, wherein the amount of the short p217+tau measured is above the LLOQ of the assay.

In yet another embodiment, the effectiveness of the treatment in the subject is determined by monitoring the amount of the p217+tau peptides, the ratio of the amount of short p217+tau peptides to the amount of long p217+tau peptides, or the ratio of the amount of short p217+tau peptides to the amount of total short tau peptides, before, during, or after the treatment, wherein the amount of p217+tau peptides, short p217+tau peptides and/or long p217+tau peptides are above the LLOQs of their respective assays. A decrease in values relative to baseline signals a positive response to treatment. Values can also increase temporarily in biological fluids as half-life of pathological tau in circulation is increased and/or pathological tau is being cleared from the brain.

According to a particular aspect, the tauopathy includes, but is not limited to, one or more selected from the group consisting of Alzheimer's disease (including familial Alzheimer's disease and sporadic Alzheimer's disease), frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Preferably, the tauopathy is Alzheimer's disease (including familial Alzheimer's disease and sporadic Alzheimer's disease), FTDP-17 or progressive supranuclear palsy.

Most preferably, the tauopathy is Alzheimer's disease (including familial Alzheimer's disease and sporadic Alzheimer's disease).

According to one embodiment, a method of the present application comprises (i) contacting a blood-based sample, preferably a plasma sample, with a capture antibody directed against a p217+tau epitope to capture p217+tau peptides in the sample, (ii) separately contacting the captured p217+tau peptides with a detection antibody directed against an epitope comprising amino acid residues 7 to 20, preferably after washing the captured p217+tau peptides, to thereby measure the amount of long p217+tau peptides, and/or with a detection antibody directed against an epitope comprising amino acid residues 116 to 127 of tau protein, preferably after washing the captured p217+tau peptides, to thereby measure the amount of long and short p217+tau peptides in the sample, and (iii) determining whether or not the subject is suitable for an anti-p217+tau antibody therapy based on the amount of the p217+tau peptides or the ratio of the amount of short p217+tau peptides to the amount of long p217+tau peptides, wherein the amount of the p217+tau peptides, short p217+tau and/or the long p217+tau is above the LLOQs of the respective assays.

According to a particular aspect, it is determined that a subject is suitable for an anti-p217+tau antibody therapy if the amount of p217+tau peptides in the blood-based sample, in particular, plasma sample, or the ratio of the amount of short p217+tau peptides to the amount of long p217+tau peptides in the blood-based sample or plasma sample is significantly higher than a corresponding baseline value, wherein the corresponding baseline value is above the LLOQ of the assays measuring p217+tau peptides, short p217+tau peptides and/or long p217+tau peptides, or the amount of p217+tau peptides, short p217+tau peptides and/or long p217+tau is above the LLOQ of their respective assays.

According to another particular aspect, a method of the present application comprises (i) contacting a blood-based sample, preferably a plasma sample, with a capture antibody directed against a p217+tau epitope to capture p217+tau peptides in the sample, or with a phosphorylation-independent capture antibody directed against a tau epitope between amino acids 150 and 250 of tau protein to capture total tau peptides in the sample, (ii) separately contacting the captured p217+tau peptides, or the captured total tau peptides, with a detection antibody directed against an epitope comprising amino acid residues 116 to 127 of tau protein, preferably after washing the captured p217+tau peptides or the captured total tau peptides, to thereby measure the amount of long and short p217+tau peptides, or the amount of total short tau peptides, in the sample, and (iii) determining whether or not the subject is suitable for an anti-p217+tau antibody therapy based on the ratio of the amount of short p217+tau peptides to the amount of total short tau peptides in the biological sample, wherein the amount of short p217+tau peptides is above the LLOQ of the assay.

According to one embodiment, it is determined that a subject is suitable for an anti-p217+tau antibody therapy if the ratio of the amount of short p217+tau peptides to the amount of total short tau peptides is significantly higher than a corresponding baseline value, wherein the amount of short p217+tau peptides is above the LLOQ of the assay.

In one embodiment, a method of the present application comprises (i) contacting a blood-based sample, preferably a plasma sample, with a capture antibody directed against an epitope comprising phosphorylated p217+tau to capture p217+tau peptides in the sample, (ii) separately contacting the captured p217+tau peptides with a detection antibody directed against an epitope comprising amino acid residues 7 to 20, preferably after washing the captured p217+tau peptide, to thereby measure the amount of long p217+tau peptides, and/or with a detection antibody directed against an epitope comprising amino acid residues 119 to 126 of tau protein, preferably after washing the captured p217+tau peptide, to thereby measure the amount of long and short p217+tau peptides in the sample, and (iii) determining whether or not the subject suffers from an amyloidogenic disease or is at risk of developing an amyloidogenic disease based on the amount of the p217+tau peptides. Diagnosis can be performed by comparing the amount or concentration of p217+tau peptides in a sample from the subject to corresponding predetermined threshold levels, wherein the amount or concentration of the p217+tau peptides is above the LLOQs of the assay of steps (i) and (ii).

The present application also relates to measuring p217+tau that is in complex with antibody in a blood-based sample, in particular, plasma, as well as free p217+tau in the sample that is not antibody-bound. In one embodiment, total antibody is captured using affinity techniques, followed by denaturing conditions including chaotrophs, heat-inactivation, or other protein disruption techniques. The p217+tau is separated from antibody using rpHPLC, and is measured using methods of the present application, allowing for quantification of antibody-bound p217+tau.

According to a general aspect, the invention relates to a method of monitoring a treatment with an anti-p217+tau antibody in a subject, the method comprising: the present application relates to a method of monitoring a treatment with an anti-p217+tau antibody in a subject, the method comprising: (i) obtaining a blood-based sample, in particular, plasma sample, from the subject, (ii) obtaining a semi-denatured sample from the blood-based sample containing total p217+tau, (iii) contacting the semi-denatured sample with a capture antibody directed against a p217+tau epitope to capture p217+tau peptides in the semi-denatured sample, and (iv) separately contacting the captured p217+tau peptides with a detection antibody directed against an epitope comprising amino acid residues 7 to 20, preferably after washing the captured p217+tau peptide, to thereby measure the amount of long p217+tau peptides, and/or with a detection antibody directed against an epitope comprising amino acid residues 119 to 126 of tau protein, preferably after washing the captured p217+tau peptide, to thereby measure the amount of long and short p217+tau peptides in the semi-denatured sample, wherein the amounts of p217+tau peptide, short p217+tau peptides, and/or long p217+tau peptides are measured above the LLOQs of their respective assays.

The semi-denatured sample is prepared from the blood-based sample containing p217+tau peptides by degrading antibodies and/or other blood components that interfere with binding of the capture antibody and/or the detection antibody to p217+tau peptides or interfere with detection of the detection antibody bound to p217+tau peptides, without degrading the p217+tau peptides present in the blood-based sample. In one embodiment, the semi-denatured sample is prepared by heating the blood-based sample at a predetermined temperature that denatures antibodies for a predetermined amount of time. The predetermined temperature may be from 75° C. to 100° C., from 80° C. to 90° C., or 85° C. The predetermined amount of time may be 0.1 to 30 minutes, 1 to 15 minutes, 2 to 10 minutes, 3 to 9 minutes, or 7 minutes. Following heat denaturing, the sample may optionally be cooled to a temperature that is suitably stable for the p217+tau peptide (e.g., at or below 4° C.), to stop further degradation of proteins within the semi-denatured sample. In one exemplary embodiment, the semi-denatured sample is prepared by heating the blood-based sample to 85° C. for 7 minutes and subsequently cooled in a 4° C. ice bath for 10 minutes.

According to another general aspect, the present application relates to a method of monitoring a treatment with an anti-p217+tau antibody in a subject, the method comprising: (i) obtaining a blood-based sample, in particular, plasma sample, from the subject, (ii) obtaining a semi-denatured sample from the blood-based sample containing total p217+tau, wherein the semi-denatured sample is heated to denature the antibodies in the sample, (iii) contacting the semi-denatured sample with a capture antibody directed against a p217+tau epitope to capture p217+tau peptides in the semi-denatured sample, (iv) separately contacting the captured p217+tau peptides with a detection antibody directed against an epitope comprising amino acid residues 7 to 20, preferably after washing the captured p217+tau peptides, to thereby measure the amount of long p217+tau peptides, or with a detection antibody directed against an epitope comprising amino acid residues 116 to 127 of tau protein, preferably after washing the captured p217+tau peptides, to thereby measure the amount of long and short p217+tau peptides in the semi-denatured sample, (v) calculating the amount of the antibody-bound p217+tau in the sample by subtracting the amount of the antibody-free p217+tau from the amount of the total p217+tau, (vi) calculating the ratio of the antibody-bound p217+tau to the antibody-free p217+tau, and (vii) monitoring the treatment with the anti-p217+tau antibody in the subject based on the calculated ratio, wherein the amounts of p217+tau peptide, short p217+tau peptides, and/or long p217+tau peptides are measured above the LLOQs of their respective assays.

According to a particular aspect, the effectiveness of the treatment in the subject is determined by monitoring the amount of the antibody-bound and antibody-free p217+tau peptides before, during, or after the treatment. A decrease in values of antibody-free p217+tau relative to baseline, or an increase in values of antibody-bound p217+tau relative to baseline, and therefore an increase in the ratio of the antibody-bound p217+tau to the antibody-free p217+tau relative to baseline, signals a positive response to treatment. Values of antibody-free p217+tau can also increase temporarily in blood-based fluids, such as plasma, as half-life of pathological tau in circulation is increased and/or pathological tau is being cleared from the brain.

In a further aspect of the present application, the assays and methods can be used to monitor p217+tau levels in a patient undergoing any treatment for tauopathy, including but not limited to administration of exogenous anti-tau antibodies, more specifically, anti-p217+tau antibodies, or any treatment for an amyloidogenic disease. Detection of levels of p217+tau in blood-based samples, in particular, plasma samples, may be used for numerous different purposes including use as a decision tool to determine if the dose level or dosing interval of the treatment should be increased or decreased to ensure attainment or maintenance of efficacious or safe drug levels; use as an aid in the initiation of anti-tau drug therapy by providing evidence of the attainment of minimum pK levels; and use as an indication that a patient should be excluded from or included in a clinical trial and as an aid in the subsequent monitoring of adherence to clinical trial medication requirements.

According to particular aspects, the capture antibody of methods of the present application is first bound to a solid support before contacting with a sample. The capture antibody may be provided in a diagnostic kit for measuring p217+tau in a blood-based sample (in particular, a plasma sample) pre-bound to a solid phase, such as to the wells of a microtiter dish or to magnetic beads. The detection antibody can contain or be attached to any detectable label (e.g., fluorescent molecule, biotin, etc.) which is directly detectable or detectable via a secondary reaction (e.g., reaction with streptavidin). Alternatively, a second reagent containing the detectable label can be used, where the second reagent has binding specificity for the primary antibody. In a particular embodiment, the detection antibody is biotinylated.

According to particular aspects, the amount of p217+tau peptides measured in methods of the present application can be determined using any suitable techniques known in the art, including ELISA and single molecule array platform. According to particular aspects, methods of the present application use a high sensitivity array platform, such as Quanterix Simoa or MSD S-plex, to measure the amount of p217+tau peptides in a blood-based sample (specifically a plasma sample), which has lower concentrations of p217+tau peptides compared to CSF.

In a further aspect of the present application, the assays and methods of the present application provides a bead-based assay for measuring p217+tau peptides in blood-based samples (e.g., blood, serum and/or plasma) having reduced interference caused by assay reagents and is therefore, more accurate and precise. It has been found that certain assay reagents when used to measure p217+tau peptides in blood-based samples interact in such a manner as to create interference with measurements obtained by the assay. In particular, in a bead-based assay where the capture antibody is bound to a magnetic bead before contacting with a blood-based sample, a sample diluent used in preparation of the blood-based sample is found to interfere with accuracy and precision of the assay in blood-based samples. For example, as shown in Example 7 below, the Sample Diluent obtained from the Simoa Homebrew Assay Starter Kit, cat #101351 (commercially available from Quanterix) demonstrates reduced bead count, which is believed to be caused by clumping of the magnetic beads used as substrates for the capture antibody. However, the assays and methods of the present application utilize sample diluents that reduce interference caused by bead clumping. In particular, the sample diluents of the present application comprise a nonionic surfactant. More specifically, the nonionic surfactant includes a hydrophilic polyethylene oxide chain and/or an aromatic hydrocarbon lipophilic or hydrophobic group. More particularly, the non-ionic surfactant is Triton X-100. The sample diluent may also comprise tris(hydroxymethyl)aminomethane (Tris), which is found to further reduce interference to the assay. Example 7, described further below, demonstrates that sample diluents containing Triton X-100 reduced interference observed and that Tris buffer-based sample diluents provided better reduction in interference as compared to Phosphate buffer-based sample diluents. The sample diluents of the present application may further include other suitable components that do not interfere with measurement of p217+tau in blood-based samples, such as, NaCl, Ethylenediaminetetraacetic acid (EDTA), heterophilic blocker, and/or Bovine Serum Albumin.

In another general aspect, the present application relates to a kit for detecting p217+tau from blood-based samples (e.g., blood, serum, plasma) comprising (a) a capture antibody directed against a p217+tau epitope, optionally a phosphorylation-independent capture antibody directed against a tau epitope between amino acids 150 and 250 of tau protein, (b) magnetic beads for conjugating the capture antibody thereto, (c) a sample diluent comprising a non-ionic surfactant, and (d) at least one detection antibody directed against a tau protein epitope comprising amino acid residues 7 to 20 or 116 to 127 of tau protein. The kit is used to measure the amount of p217+tau peptides, the ratio of the amount of short p217+tau peptides to the amount of long p217+tau peptides, and/or the ratio of the amount of short p217+tau peptides to the amount of total short tau peptides in a sample.

In another aspect of the present application, p217+tau measurements obtained from blood-based samples (e.g., blood, serum, plasma) according to the assays and methods described above are further analyzed in a computing device to detect and/or predict tauopathy in a subject. In particular, the p217+tau measurements obtained from blood-based samples are analyzed by a computing device in combination with data corresponding to measurements obtained for other biomarkers that are also detectable from blood-based samples to provide further improved detection and/or prediction of tauopathy in the subject. The improved ability to detect and/or predict tauopathy, specifically AD, using biomarker(s) that can be adequately measured from blood-based samples can be used for various diagnostic purposes, e.g., for diagnosing AD or other tauopathies in a subject, monitoring the effectiveness of a treatment, identifying a subject suitable for an anti-tau or anti-p217+tau treatment, pre-screening subjects for PET imaging and/or CSF assays for further detection of AD or other tauopathies, identification of subjects for enrollment in clinical trials relating to AD or other tauopathies, etc.

In one exemplary embodiment, a method for detecting or predicting tauopathy in a subject is provided. The method comprises detecting an amount of p217+tau peptides in a blood-based sample (e.g., blood, serum, plasma) using an assay. The assay may be any of the exemplary assays described in the present application. In particular, the assay measures the amount of p217+tau peptides in blood-based samples, in particular plasma, by contacting the sample with a capture antibody which binds to p217+tau peptides in the sample and in a separate step, contacting the captured p217+ peptides with an anti-tau detection antibody, which is labeled with a reporter element that allows detection of the captured p217+tau species. More particularly, the assay detects an amount of p217+tau peptides in a plasma sample by contacting the plasma sample with a capture antibody directed against a p217+tau epitope to bind the capture antibody to p217+tau peptides in the plasma to form antibody-peptide complexes, and separately contacting the antibody-peptide complexes with a detection antibody to bind the detection antibody to the antibody-peptide complexes.

A computing device obtains the p217+tau measurements detected by the assay to generate tau data corresponding to the amount of p217+tau peptides. The tau data may represent the amount of p217+tau peptides detected by the assay. Alternatively, the tau data may represent a binary status (yes/no) indicating whether the amount of is above a predetermined threshold value. The assay is sufficiently sensitive, as discussed above, such that the predetermined threshold value is above a LLOQ of the assay method. The computing device may also obtain medical data of the subject, such as, for example, demographic information (e.g., age, sex), medical history, Electronic Medical Records (EMR), pharmacy data corresponding to the patient's medication records, etc. In particular, the computer device may obtain biomarker data corresponding to measurement or binary status for at least one biomarker detected from the patient. The biomarker may be any suitable biomarker for tauopathy. Preferably, the biomarker is detectable from blood-based samples, in particular, plasma samples, of a subject. For example, the biomarker may be selected from a group consisting of amyloid-O (Aβ), neurofilament light chain (NFL), adiponectin, leptin, and other inflammatory or metabolic markers. More specifically, the biomarker is selected from NFL, adiponectin and leptin. The computing device analyzes the tau data and the biomarker data using a machine learning module to determine or predict whether the subject suffers from tauopathy or is at risk of developing tauopathy. The machine learning module is trained using a set of reference data. The machine learning module compares the tau data and the biomarker data to a set of reference data to determine or predict whether the subject has tauopathy or is at risk of developing tauopathy. The set of reference data includes tau data and biomarker data, along with data corresponding to brain pathology of tauopathy (e.g., stage of disease, amount of p217+tau detected in CSF, PET measurements of tau in brain tissue, etc.), for a reference group of patients.

The machine learning module may be a supervised and/or unsupervised machine learning module. The machine learning module may be a machine learning classifier, for identifying dataset as correlating to one of two categories. The machine learning module may include support vector machine, random forest, logistic regression, gradient boosting module, or ensemble modules thereof. In one embodiment the machine learning module is an ensemble module comprising at least one of support vector machine, random forest, logistic regression, and/or gradient boosting module.

Those skilled in the art will understand that the exemplary computer-implemented embodiments described herein may be implemented in any number of manners, including as a separate software module, as a combination of hardware and software, etc. For example, the exemplary methods may be embodiment in one or more programs stored in a non-transitory storage medium and containing lines of code that, when compiled, may be executed by one or more processor cores or a separate processor. A system according to one embodiment comprises a plurality of processor cores and a set of instructions executing on the plurality of processor cores to perform the exemplary methods discussed above. The processor cores or separate processor may be incorporated in or may communicate with any suitable electronic device, for example, on board processing arrangements within the device or processing arrangements external to the device, e.g., a mobile computing device, a smart phone, a computing tablet, a computing device, etc., that may be in communications with at least a portion of the device.

### EXAMPLES

The following examples are to further illustrate the nature of the invention. It should be understood that the following examples do not limit the invention and that the scope of the invention is to be determined by the appended claims.

### Example 1: High Sensitivity Assay for Detecting P217+tau in Plasma

An exemplary embodiment of the improved assay of the present application is provided in Example I. The exemplary embodiment of Example I utilizes a bead-based enzyme-linked immunosorbent assay (ELISA) to detect and/or quantity the presence of p217+tau peptides in a sample. Specifically, Example I utilizes the Single Molecule Array (SiMoA) bead-based digital ELISA system available from Quanterix Corp. (Boston, MA). The SiMoA assay uses arrays of femtoliter-sized reaction chambers to digitally count individual immunocomplexes. Assay-specific regents were prepared, as discussed further below, and provided to a SiMoA analyzer to react with and detect p217+tau peptides in samples. The assay-specific reagents include: paramagnetic capture beads having a 2.7 μm diameter, buffers and reagents from the Simoa Homebrew Assay Starter Kit, cat #101351 (commercially available from Quanterix), Wash Buffer 1 (commercially available from Quanterix), magnetic beads (commercially available as Simoa Homebrew Helper Bead Vial (918), cat #101732 from Quanterix), a capture antibody, and a detection antibody. The capture antibody of Example 1 is a pT3 mouse monoclonal antibody (mAb). The detection antibody of Example 1 is hT43 mAb.

Each sample analyzed in Example I is diluted in a sample diluent. The exemplary sample diluent used in Example I includes 50 mM Tris buffer, 100 mM NaCl, 5 mM EDTA, 2% (v/v) Bovine Serum Albumin, and 0.5% (v/v) Triton X-100, the Heterophilic Blocking Agent HBR-9 (commercially available as cat #3KC564 from Scantibodies Laboratory, Santee CA). The sample diluent has a pH of 7.4.

The assay of Example I was calibrated using a calibrant peptide custom made by New England Peptide. The calibrant peptide is peptide containing hT43 and pT3 epitopes connected by a PEG4 linker and has a molecular weight of 4357 g/mol. The calibrant peptide has an amino acid sequence of SEQ ID NO: 18.

### Reagent Preparation

In the first step, the paramagnetic captures beads were coated with 0.3 mg/mL of a capture antibody, which in Example I is pT3 mAb, following the protocol provided in the Quanterix manual to attach the capture antibody to the beads. The coated capture beads were diluted in a Bead Diluent Buffer from the Simoa Homebrew Assay Starter Kit to 100,000 beads/mL, and 300,000 beads/mL Helper Beads were added to obtain a total concentration of beads of 400,000 beads/mL.

The detection antibody, which in Example I is hT43 mAb, was biotinylated at 60× following the protocol provided in the Quanterix manual and diluted in the sample diluent described above to for a detection solution having a concentration of 0.9 μg/mL of the detection antibody.

Streptavidin β-D-galactosidase (SBG) concentrate was diluted to 200 pM in the SBG diluent from the Simoa Homebrew Assay Starter Kit.

The calibrant peptide was reconstituted to 5 mg/mL in 0.1% phosphoric acid/water, aliquoted to units of 20 μL each and frozen. When ready for use, the calibrant peptide aliquot units were thawed and diluted 1:1000 (e.g., 1.5 ul into 1498.5 ul), and the dilutions were further diluted 1:1000 with the sample diluent so that the final concentration of the peptides was 5000 μg/ml. The assay of Example I was calibrated with varying concentrations of the calibrant peptide to form a standard curve across the following concentrations: 30, 10, 3.33, 1.11, 0.37, 0.186, 0.093, 0.046, 0.023, 0.012, 0.006 and 0 pg/mL.

Plasma samples analyzed by the assay of Example I were diluted 1:2 in the sample diluent.

### SiMoA Assay

A custom SiMoA assay was created comprising a three-step protocol. This three-step protocol includes the steps of: contacting a sample for analysis with the pT3mAb attached capture beads, as prepared above, with a sample for analysis for 35 minutes, followed by washing the capture beads with a Phosphate-buffered saline (PBS) Tween-20 solution, in particular, the Wash Buffer 1 designed for the Simoa HD-1 instrument (commercially available from Quanterix), then incubating the capture beads with the detection antibody for 5 minutes, followed by washing the capture beads for a second time with Wash Buffer 1, incubating the capture beads with SBG for 5 minutes, washing the beads again with a PBS-based solution, in particular Wash Buffer 1, and finally adding 25 μL of 100 μM resorufin-β-D-galactopyranoside (RGP) prior to loading into measurement discs for imaging and measurements by the SiMoA HD-1 instrument. Each reaction was performed in Simoa cuvettes and comprised 25 μL of the beads solution comprising the pT3 mAb attached paramagnetic beads and the Helper Beads as prepared above, 172 μL of a diluted sample or calibrant, 100 μL of the detection solution comprising the detection biotinylated detection antibody as prepared above, 100 μL of SBG solution, and 25 μL of 100 μM RGP.

The SiMoA analyzer utilizes high-resolution fluorescence imaging to detect the fraction of beads its arrays of femotoliter-sized reaction chambers that fluoresces corresponding to the fraction of beads associated with at least one enzyme, and the fluorescence intensity of each reaction chamber. The SiMoA analyzer generates an output for an average number of enzymes per bead (AEB) based on these measurements.

### Example 2: Detection of Assay-Competing Antibodies Present in Plasma

With additional upstream sample manipulation, the high sensitivity of Example 1 can be used to measure the levels of p217+tau even in presence of assay-competing antibodies that compete with the reagents of the assay of Example 1, such as, for example, those assay-competing antibodies that are endogenously produced within a subject or assay-competing antibodies exogenously administered to the subject. The method described herein in Example 2 can be used as pharmacodynamic assay to study therapeutic anti-p217+tau antibodies such as humanized pT3 mAb present in plasma. For example, method of Example 2 can be used to measure impact of an active agent for treating tauopathy, specifically an anti-p217+tau monoclonal antibody (e.g., humanized pT3 mAb), on peripheral levels of tau present in a plasma sample retrieved from a human subject.

An aliquot of a plasma sample was first diluted 1:3 in 0.1 M NaOAc, and subsequently heated at 85° C. for 7 minutes, followed by chilling in an ice bath (4° C.) for 10 minutes. The heated and subsequently chilled sample fluid was centrifuged at 14000×g for 10 minutes at 4° C. and the supernatant was separated from the precipitate. A 1 M Tris Base solution was added to the supernatant at 7% volume to achieve neutral pH of the supernatant, and obtain an exemplary semi-denatured sample. In parallel, a second aliquot of the plasma sample was chilled in an ice bath for the duration for the duration of the preparation of the semi-denatured sample fluid. This second aliquot is also referred in Example 2 herein as a non-denatured sample fluid.

Output generated by the SiMoA analyzer for the semi-denatured fluid signal corresponds to a total amount of p217+tau present in the plasma sample, while output generated for the non-denatured fluid corresponds free p217+tau in the plasma sample. The outputs are correlated to the standard curve from the calibrant peptide to obtain concentrations of the total amount of p217+tau and free p217+tau present in the plasma sample, respectively. Subtracting the latter from the former provides a quantitative measurement of an amount of free p217+tau present in the plasma sample.

The precise heating time and temperature used in Example 2 was determined, using CSF and exogenously added antibodies, as a combination of heating time and temperature that is sufficient to irreversibly modify any interfering antibodies in a sample such that they would no longer interfere binding of p217+tau antibodies in an assay, while the p217+tau signal itself was spared any impact. In particular, data obtained using CSF and exogenously added antibodies showed that p217+tau signal is resistant to 85° C. heat for at least 10 minutes, yet antibodies are denatured after only 2 minutes of 85° C. heat. Therefore, the results obtained using the semi-denatured fluid and non-denatured fluid of Example 2 do not provide a direct measure of whether the capture antibodies are bound to p217+tau, but rather demonstrates that assay-competing antibodies, that interfere with the ability of a p217+tau assay to detect and quantify an amount of p217+tau, are present in plasma.

### Example 3: Prior p217+tau Assay not Suitable for Use with Plasma

As discussed above, an assay for measuring p217+tau peptides in biologic samples was previously disclosed in the Kolb '492 patent. However, the Kolb '492 patent does not provide any examples for quantifying amounts of p217+tau peptides present in human serum or plasma. Instead, the Kolb '492 patent indicated that crude serum or plasma samples have difficulties with interferences and cannot be measured and quantified with sufficient sensitivity until the samples were immunoprecipitated followed by heat denaturing of the elute.

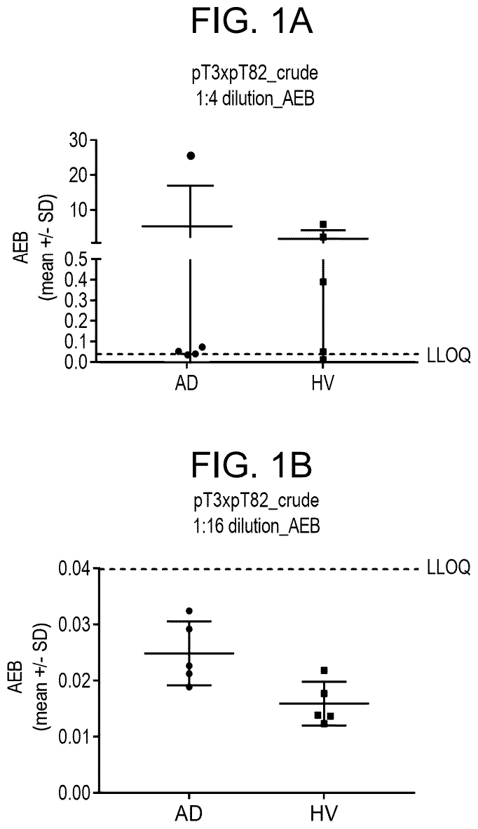

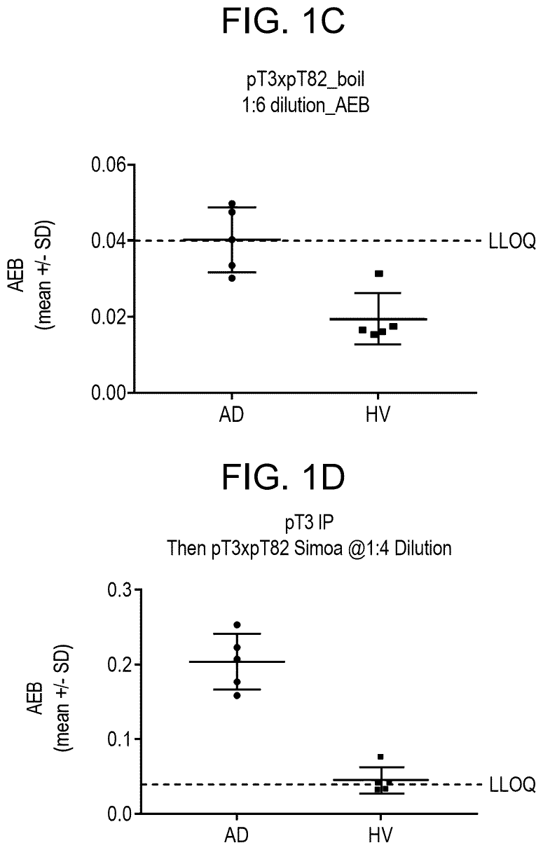

Example 3 evaluates the assay described in Example 1 of the Kolb '492 patent using a set of human plasma samples obtained from 5 healthy volunteer (HV) control subjects and 5 subjects known to have Alzheimer's Disease (AD). pT3 mAb is used as the capture antibody and pT82 mAb is used as the detection antibody in this example. Data obtained according to Example 3 are shown in FIGS. 1a-1d in units of AEB, as generated by the SiMoA analyzer. The left side of each graph shows data corresponding to AD subjects and the right side of the graph shows data corresponding to HV. For each category of subjects, a mean value is shown as the longer horizontal line, with ±standard deviation (SD) of the dataset shown with the shorter lines above and below the mean value line.

Each of these plasma samples were diluted according to the Kolb '492 patent at two different dilutions—1:4 and 1:16. The results obtained using both plasma sample dilutions are provided in FIGS. 1a and 1b, respectively. The data shown in FIG. 1a demonstrates that at 1:4 dilution, 40% of the plasma samples measured below the LLOQ of the assay described in Example 1 of the Kolb '492 patent while 20% measured at the LLOQ and another 40% measured significantly higher than all the other samples. In particular, at 1:4 dilution 6/10 samples of Example 3 measured at or below LLOQ (AEB=signal=0.035=S/N>2 and CV<20%), the other 4 samples presented markedly higher signal (11-745× above LLOQ). As shown in FIG. 1b, at 1:16 dilution, all of the plasma samples, including those 40% that had measured significantly higher at 1:4 dilution, measured below the LLOQ of the assay described in Example 1 of the Kolb '492 patent. More particularly, at 1:16 dilution all samples of Example 3 measured below LLOQ, indicating that the high signal at 1:4 dilution in the 4/10 subjects of Example 3 as noted above was substantially non-linear and thus can be considered as an artifact from the plasma matrix. The data shown in FIGS. 1a and 1b demonstrates that there is a lack of sensitivity and a lack of dilution linearity in detecting p217+tau peptides in plasma using the assay described in Example 1 of the Kolb '492 patent and therefore, such an assay is not suitable for analyzing an amount of p217+tau peptides present in plasma.

A set of plasma samples from the same 5 HV and 5 AD subjects were denatured according to Example 2 above to obtain 10 different semi-denatured sample fluids. The process of obtaining semi-denatured sample fluids as described in Example 2 modifies interfering antibodies such that they would no longer interfere with binding of p217+ antibodies to p217+tau peptides in the sample, without degrading the p217+tau signal detected by the p217+ antibodies. Each of the semi-denatured sample fluids are diluted 1:6 and measured using the assay described in Example 1 of the Kolb '492 patent. The results are shown in FIG. 1c. As can be seen in FIG. 1c, the process of obtaining a semi-denatured sample fluid abolished all quantifiable signal in 40% of samples that had previously measured significantly higher than LLOQ at 1:4 dilution without any denaturing step (shown in FIG. 1b). The process of obtaining semi-denatured sample fluid as described in Example 2 does not degrade the p217+tau signal detected by the p217+ antibodies. Therefore, the data shown in FIG. 1c suggest that the signal detected from plasma shown in FIG. 1a may be contaminated with interference and/or artifacts from other components other than p217+tau peptides. In particular, the high p217+tau signal in the 4/10 plasma samples shown in FIG. 1a was eliminated after denaturing, indicating this signal is not true tau signal. The 1:16 crude plasma data shown in FIGS. 1a and 1c indicated higher signal in AD vs HV, demonstrating that steps to eliminate matrix interference can reveal biomarker relevant p217+tau signal in plasma. However, poor sensitivity precludes the assay described in Example 1 of the Kolb '492 patent from being suitable for measuring p217+tau in plasma samples.

The Kolb '492 patent acknowledged that crude serum or plasma may be plagued by sensitivity and matrix interference hurdles and describes that an enrichment strategy using immunoprecipitation may be used in combination with the assay described in Example 1 of the Kolb '492 patent to provide blood-based measurements of pathological tau. The following demonstrates that immunoprecipitation in combination with the assay described in Example 1 of the Kolb '492 patent provides improved sensitivity and separation of HV from AD subjects.

A set of plasma samples from the same 5HV and 5AD subjects were immunoprecipitated for its p217+tau signal using pT3 antibodies prior to measuring according to the assay described in Example 1 of the Kolb '492 patent. The immunoprecipitated samples were diluted with the sample diluent in a ratio of 1:4. Results from these immunoprecipitated samples having a 1:4 dilution are shown in FIG. 1d. Comparing the results shown in FIG. 1d to FIG. 1a, which is measured at the same dilution ratio but without first immunoprecipitation with pT3 antibodies, the immunoprecipitations steps improved sensitivity of the assay such that plasma samples from AD subjects all measure within linear range, and with good separation from HV samples. FIG. 1d shows that the amount of p217+tau peptides present in plasma obtained from HV samples are slightly above the LLOQ of the assay described in Example 1 of the Kolb '492 patent, but does not provide reliably quantifiable results above the LLOQ of the assay (e.g., the LLOQ falls within the standard deviation for HV samples). These results indicated that purification and concentration of the plasma p217+tau signal could yield a useful plasma p217+tau assay. However, immunoprecipitation is a laborious and imprecise process, which is unduly burdensome and may introduce further inaccuracies into an assay. The assays and methods of the present application do not require a separate immunoprecipitation step to concentrate its p217+tau signal in order to obtain results having sufficient sensitivity for detection of p217+tau peptides in human plasma samples.

### Example 4: Comparison of Prior Assay to the High Sensitivity Assay of the Present Application for Detecting p217+tau Peptides

The assays and method of the present application includes separate steps for contacting a plasma sample with a capture antibody to bind the capture antibody to p217+tau peptides in the plasma to create antibody-peptide complexes, and contacting the antibody-peptide complexes with a detection antibody to bind the detection antibody to the antibody-peptide complexes after a wash, which is exemplified in Example 1. The first step of contacting the plasma sample with the capture antibody and subsequently washing the antibody-peptide complexes before contact with the detection antibody separates the p217+tau signal from interfering components in the plasma sample. In particular, p217+tau peptides in the sample are bound to the capture antibody while interfering components of the sample are washed away before the detection antibody is added to the antibody-peptide complexes. Assays and methods including these separate steps are also referred herein as “3-step” assays. In contrast, the assay described in Example 1 of the Kolb '492 patent combines its biologic sample with both the capture antibody and the detection antibody at the same time to allow binding of both antibodies to the biologic sample before a washing step. The assay as described in Example 1 of the Kolb '492 patent is also referred herein as “2-step” assays. In this example, the incubation time and sample volume input in the first step of the “3-step” assay was also increased, as compared to the “2-step” assay, to allow maximal capture of signal. As demonstrated further below in Example 4, it has been surprisingly found that the “3-step” assay of the present application provides improved sensitivity over the ‘“2-step” assay when measuring p217+tau peptides from serum, which is not observed when measuring from CSF.

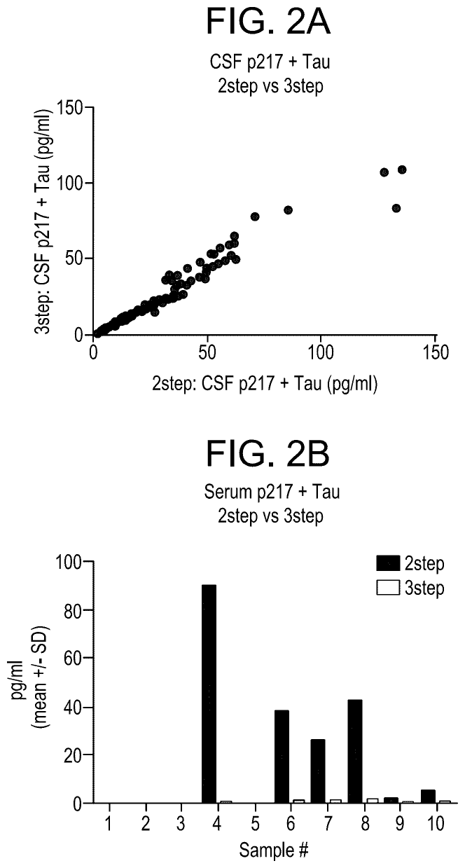

Example 4 compares the “3-step” assay of the present application to the “2-step” assay measuring p217+tau peptides from both CSF and serum. A set of CSF samples for a plurality of subjects having varying cognitive states were measured using both the “2-step” and “3-step” assays. Specifically, 96 CSF samples from 21 mild-moderate dementia subjects in a clinical study were measured using both the “2-step” and “3-step” assays. For each sample, the results (in pg/mL of p217+tau detected) obtained using the “2-step” assay (shown in the X-axis) is mapped to the result obtained using the “3-step” assay (shown in the Y-axis) in FIG. 2a. In addition, a set of serum samples for the same 5HV and 5AD subjects as Example 3 were measured using both the “2-step” and “3-step” assays. The results (in pg/mL of p217+tau detected) obtained are shown in a bar graph in FIG. 2b, where the left side bar for each sample corresponds to result obtained using the “2-step” assay and the right side bar corresponds to result obtained using the “3-step” assay.

As can be seen in FIG. 2a, the “2-step” and “3-step” assays demonstrated high correlation (r²=0.94) when used to measure p217+tau peptides in CSF sample. However, FIG. 2b shows that such a correlation is not observed when the assays are used to measure p217+tau peptides in serum samples. Furthermore, although FIG. 1a showed a subset of samples providing measurements that are much higher than the remainder of the samples when the “2-step” assay is used to measure p217+tau peptides from plasma, FIG. 2b shows that these outlier high signals are dramatically reduced when the “3-step” assay is used to measure p217+tau peptides from serum. The data of FIG. 2b shows that the outlier high p217+tau signals observed using the “2-step” assay were not similarly observed with the same samples measured using the “3-step” assay. Therefore, the data of FIG. 2b demonstrates that the outlier high p217+tau signals observed using the “2-step” assay correlates to interference and/or artifact of the assay and does not accurately measure the p217+tau peptides present in the sample. This data shows that there is negligible matrix interference in CSF, but substantial positive interference in blood products.

### Example 5: Comparison of Detection Antibodies Using the High Sensitivity Assay of the Present Application for Detecting p217+tau Peptides

Example 5 evaluates concordance and relative fragmentation between three types of biologic fluids, i.e., CSF, serum and plasma, using two different detection antibodies with the exemplary assay as described in Example 1. Specifically, Example 5 compares the difference between hT43 and pT82 as detection antibodies for use in the assay of Example I (except for the different detection antibodies as specified in Example 5). The capture antibody is pT3 for both exemplary embodiments. Therefore, the two exemplary assays compared in Example 5 are pT3xhT43 and pT3xpT82.

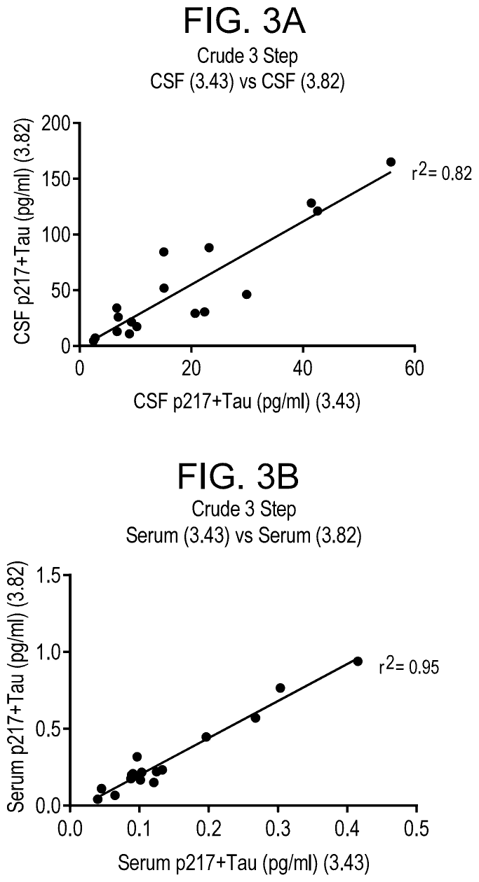

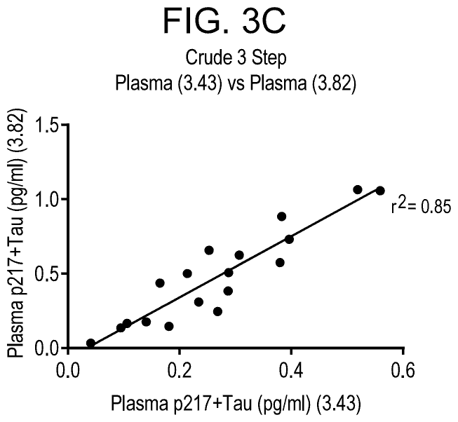

CSF, serum and plasma from 18 AD (in particular, those having mild-moderate dementia) subjects in a clinical study were measured using the assay of Example 1 and a modified assay similar to Example 1, except the detection antibody is modified to hT43 mAb. The results (in pg/mL of p217+tau detected) obtained are shown in FIGS. 3a, 3b and 3c for CSF, serum and plasma, respectively. For each type of biologic fluid, the results (in pg/mL of p217+tau detected) obtained using the pT3xhT43 assay (shown in the X-axis) is mapped to the result obtained using the pT3xpT82 assay (shown in the Y-axis) in FIGS. 3a, 3b and 3c for CSF, serum and plasma, respectively. A linear regression line, along with a R² value for the linear regression line is shown in each of FIGS. 3a-3c. As shown in FIGS. 3a-3c, in all three sample types, results obtained using the hT43 detection antibody and the pT82 antibody were highly concordant (R²=0.82 to 0.95). The slopes of the linear regression line shown in FIGS. 3a-3c are 2.83, 2.41 and 2.05, respectively. In addition, the pT3xpT82 assay obtained results at ˜2.5× higher levels than the pT3xhT43 assay across CSF, serum and plasma. As discussed above, pT3 recognizes an epitope at amino acids 210-220 of the human tau protein. The detection antibody hT43 recognizes amino acids 7-20 of the human tau protein, and the detection antibody pT82 recognizes amino acids 116-127 of the tau protein. Therefore, pT82 recognizes an epitope closer to pT3 than that recognized by hT43, which allows pT82 to recognize short p217+ peptide fragments in addition long p217+ peptide fragments as compared to hT43. It is believed that the pT3xpT82 assay reports higher concentrations than the pT3xhT43 assay because tau is known to be highly fragmented in CSF and that pT82 has to ability to detect shorter, and hence more, p217+ peptide fragments than hT43. Interestingly, similar higher levels for the pT3xpT82 assay is observed also in serum and plasma, indicating that crude fragmentation pattern (between amino acids 20 and 116) of tau may be similar in blood components (e.g., serum and plasma) as in CSF, and that there is not greater fragmentation between the hT43 and pT82 epitopes in blood products. Because the pT3xhT43 and pT3xpT82 assays are shown to be highly concordant across CSF, serum and plasma suggesting that the p217+tau fragmentation level reflects fragmentation in CSF, the two assays evaluated in Example 5 are believed to be interchangeable. However, the pT3xhT43 assay provides greater sensitivity over the pT3xhT43 assay.

### Example 6: Comparison of Different Sample Diluents for Use in Assay for Detecting p217+tau Peptides

Example 6 evaluates the different sample diluents for use in the assay of Example I (except for the different sample diluent as specified in Example 6) to determine which diluent would reduce bead clumping and/or artifacts in the p217+tau signal obtained by the assay. The impact of buffer type (PBS vs Tris), NaCl concentration, and presence of heterophilic blocker (e.g. blocking human anti-mouse interactions) was measured in terms of impact on artifact serum p217+tau signal and on number of beads detected at the end of the ELISA method. The different sample diluents evaluated in Example 6 are shown below in Table 1.

TABLE 1

| Sample | |
|---|---|
| Diluent | Composition |
| 1 | Sample Diluent from the Simoa Homebrew Assay Starter Kit |
| 2 | 50 mM Phosphate buffer, 50 mM NaCl, and 0.5% (v/v) Triton |
| | X-100 |
| 3 | 50 mM Phosphate buffer, 100 mM NaCl, and 0.5% (v/v) Triton |
| | X-100 |
| 4 | 50 mM Tris buffer, 50 mM NaCl, and 0.5% (v/v) Triton X-100 |
| 5 | 50 mM Tris buffer, 100 mM NaCl, and 0.5% (v/v) Triton X-100 |
| 6 | 50 mM Phosphate buffer, 50 mM NaCl, 0.5% (v/v) Triton |
| | X-100, and HBR-9 |
| 7 | 50 mM Phosphate buffer, 100 mM NaCl, 0.5% (v/v) Triton |
| | X-100, and HBR-9 |
| 8 | 50 mM Tris buffer, 50 mM NaCl, 0.5% (v/v) Triton X-100, |
| | and HBR-9 |
| 9 | 50 mM Tris buffer, 100 mM NaCl, 0.5% (v/v) Triton X-100, |
| | and HBR-9 |

Although not specified in Table 1, each Sample Diluent also includes 5 mM EDTA and 2% (v/v) Bovine Serum Albumin.

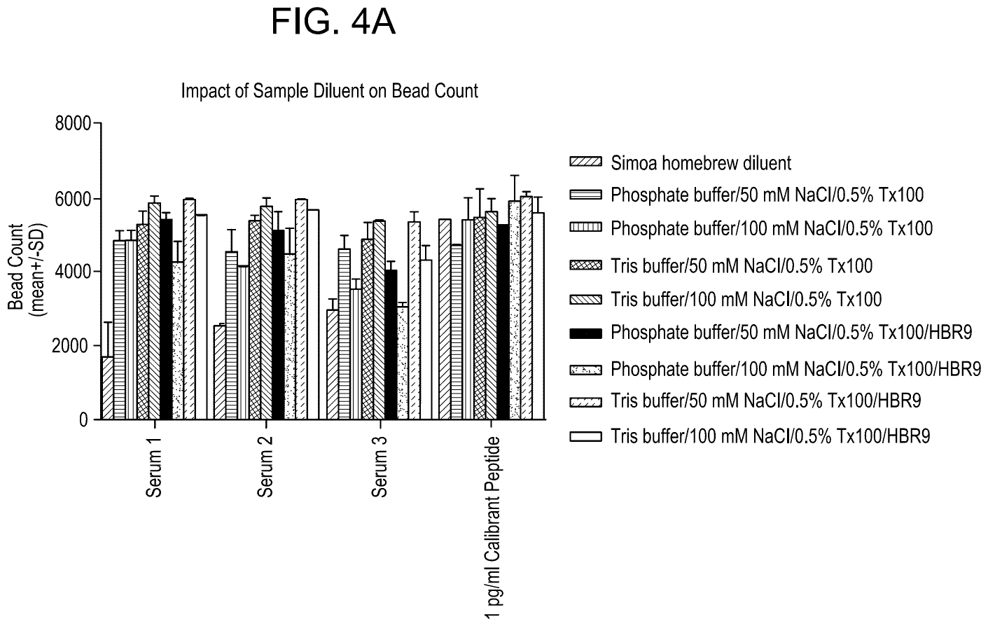

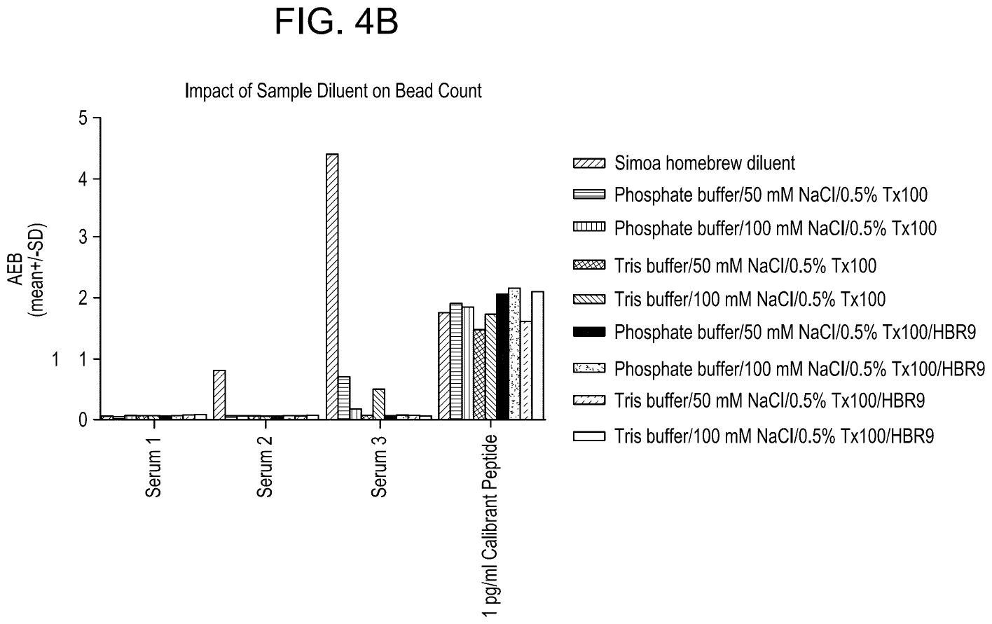

Three pooled serum samples obtained from human subjects having high tau levels and a 1 pg/mL solution of the calibrant peptide (as negative control) were analyzed using a modified assay similar to Example 1 with the different sample diluents as described herein in Example 6. The SiMoA analyzer was used to determine the number of beads loaded onto the SiMoA disc and the AEB of each combination of sample and diluent. FIG. 4a shows the number of beads loaded onto the SiMoA disc for the three serum samples and the calibrant peptide in each of the 9 different sample diluents. FIG. 4b shows fluorescent signals detected by the SiMoA analyzer in units AEB for the three serum samples and the calibrant peptide in each of the 9 different sample diluents. FIGS. 4a and 4b provide data represented as bar graphs for each sample measured in the different sample diluents, with are shown in the same order as listed in Table 1.

As can be seen in FIG. 4a, Sample Diluent 1, which is the sample diluent from the Simoa Homebrew Assay Starter Kit provides a significantly lower bead count in all three serum samples as compared to the calibrant peptide solution (1688-2921 vs. 5378). Sample Diluents 2-9 improved the beads counts in serum, and substantially reduced artifact signal seen in two of the samples measured using the Simoa Homebrew diluent (Sample Diluent 1). It is believed that this reduced bead count observed in serum is caused by clumping of the paramagnetic beads used as substrates for the capture antibodies in the assay, when the assay is used to analyze serum samples. Clumping of the paramagnetic beads is undesired as it negatively impacts the accuracy and precision of the assay for detecting p217+tau in serum. The data shown in FIG. 4a demonstrates that sample diluents 2-9, all of which include the detergent Triton X-100, provide increased bead count and therefore, reduced bead clumping that would otherwise negatively interfere with the accuracy and precision of the assay. Furthermore, FIG. 4a shows that Tris buffer-based sample diluents provided higher bead counts than phosphate-based buffers, demonstrating that Tris based buffers are useful in reducing interference to assay accuracy and precision caused by bead clumping. As can be seen in FIG. 4b, all of the sample diluents provided similar levels of fluorescent signals for the calibrant peptide solution. However, Sample Diluent 1 also resulted in significant fluorescent signals detected, which were not observed with the other sample diluents (although Sample Diluents 2, 3 and 5 also resulted in detection of some fluorescent signal). These increased fluorescent signals observed with Sample Diluent 1 are believed to be correlated to interference and/or artifacts of the assay, similar to that demonstrated for the “2-step” assay in FIG. 2b, and does not accurately measure the p217+tau peptides present in serum samples. FIG. 4b also shows that the addition of HBR-9, which is a heterophilic blocker designed to reduce anti-mouse IgG bridging of assay reagents, further reduces interference and/or artifacts in the assay. Specifically, as can be seen in FIG. 4b, Sample Diluents 6-9 showed less fluorescent signal than Sample Diluents 2-5 with one of the serum samples indicating that the addition of HBR-9 provides further reduction of interference and/or artifacts to the assay. The data in FIGS. 4a and 4b show that of the Sample Diluents evaluated in Example 4, Sample Diluent 9, which is the same as the sample diluent described in Example 1, provided both minimal bead clumping and artifact signal. In view of the data discussed above, particular improvements were observed when using Tris buffer, lower NaCl concentration and heterophilic blocker.

### Example 7: Comparison of Detection of p217+tau Peptides in Serum and Plasma

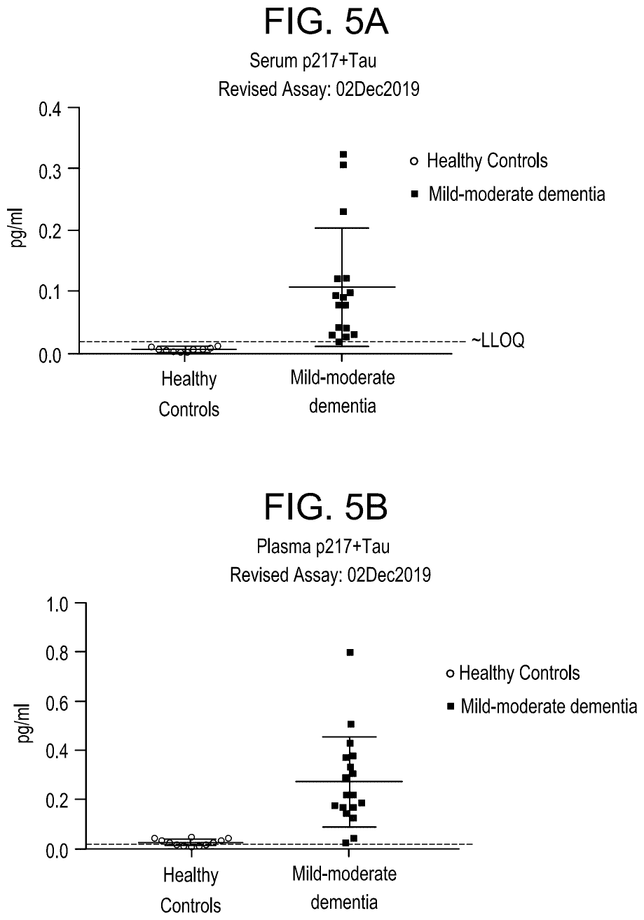

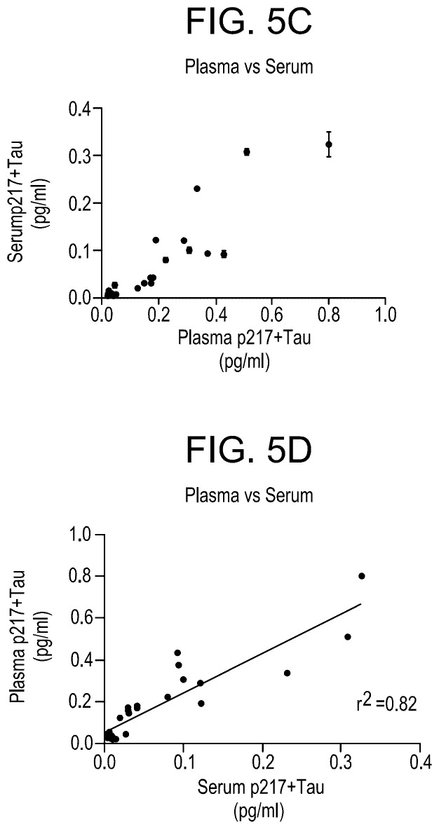

Example 7 evaluates detection of p217+tau peptides in serum and plasma using the exemplary assay described in Example 1. A set of serum samples from 10 HV (also referred herein as Healthy Volunteer or Healthy Control) and the same 16 AD subjects as Example 5 were measured using the exemplary assay of Example 1 and the results are shown in FIG. 5a. Samples from HV subjects were obtained from a blood collection service and presumed to be cognitively normal. A set of plasma samples from a subset of 12 HV and 18 AD subjects from the group of subjects of FIG. 5a were measured using the exemplary assay of Example 1 and the results are shown in FIG. 5b. In FIGS. 5a and 5b, the left side of each graph shows data corresponding to HV subjects and the right side of the graph shows data corresponding to AD subjects. For each category of subjects, a mean value is shown as the longer horizontal line, with ±standard deviation (SD) of the dataset shown with the shorter lines above and below the mean value line. A dotted line is also shown across each of FIGS. 5a and 5b to show the LLOQ of the assay for each of serum and plasma, respectively. Data obtained according to Example 7 are shown in FIGS. 5a-5b in pg/mL. In addition, for each subject where both plasma and serum samples were reported in FIGS. 5a and 5b (i.e., 10 HV and 16 AD subjects), the results (in pg/mL of p217+tau detected) obtained using plasma obtained from the subject are mapped to the results obtained using serum from the same subject in FIGS. 5c and 5d. As can be seen in FIGS. 5a and 5b, both serum and plasma samples obtained from AD subjects measured significantly higher than serum and plasma sampled obtained from HV subjects. This data suggests that both serum and plasma may be useful for diagnostic purposes. Surprisingly, measurements obtained from plasma reported ˜2-3× (specifically 2.3×) higher concentration than measurements obtained from serum, as determined by averaging ratios of p217+tau measured from plasma to p217+tau measured from serum determined for all the subjects. However, as shown in FIG. 5d, a linear regression of the data shows a slope of 1.9, indicating that measurements obtained from plasma is 1.9× higher than measurements obtained from serum. Because the detectable level of p217+tau analyte is low in serum, many of the serum samples obtained from HV subjects measured below the LLOQ of the assay. However, because detection of p217+tau in plasma is higher, all of the plasma samples obtained from HV & AD patients measured at or above the LLOQ of the assay. Therefore, the exemplary assay described in Example 1 is useful for detecting and quantifying an amount of p217+tau peptides in plasma for both HV and AD subject. In addition, as can be seen in FIG. 5b, the range of p217+tau peptides detected from HV subjects do not substantially overlap with the range of p217+tau peptide detected from AD subjects. All of the serum samples obtained from HV patients measured below the LLOQ of the assay (shown in FIG. 5a), but a majority (11 of 12) of plasma samples from the same HV patients measured above the LLOQ of the assay in linear range (shown in FIG. 5b). The plasma and serum p217+tau concentrations measures correlated well (r²=0.82), however the plasma measurements were on average ˜1.9× higher than those in serum, as shown in FIG. 5c. Therefore, the assay of the present application surprisingly provides quantitative data that can be used to separate HV subjects from AD subjects. Specifically, a subject may be determined to have or is at risk of developing tauopathy, in particular, Alzheimer's Disease, when the amount of p217+tau peptides detected in plasma is above a predetermined threshold value (e.g., ˜0.1 pg/mL based on the data shown in FIG. 5b).

### Example 8: Linear Range of Assay for Detecting p217+tau in Plasma

Linear Range with Calibrant Material

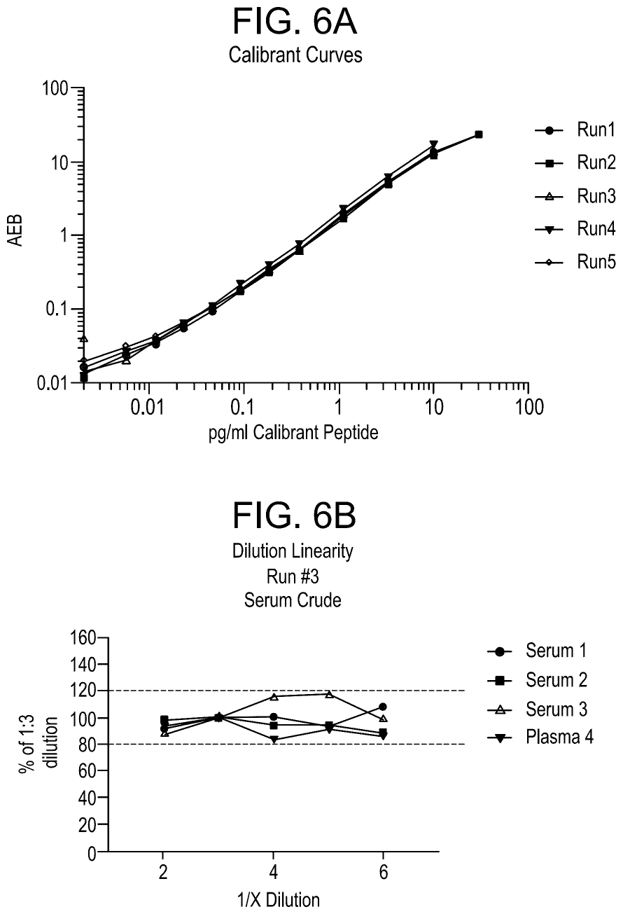

Calibrant peptides described in Example 1 were produced. The calibrant peptides contained the core epitopes of pT3 and hT43, separated by a PEG4 linker, and were used to generate standard curves correlating outputs of AEB from the SiMoA analyzer to concentrations of calibrant peptides. Representative standard curves generated by 5 separate runs of the different dilutions of the calibrant peptides as specified in Example 1 are shown in FIG. 6a. A 4-parameter curve fit data reduction method (4PL, 1/y2 weighted) was used to generate the calibration curves. The lower limit of detection (LLOD) of the exemplary assay of Example 1 was determined as the calculated calibrant level yielding an AEB equal to the average of the zero calibrator+2.5 standard deviations (SD), including 10% CV. With these criteria, the representative data yielded an LLOD of ˜0.002 pg/mL. The linear range of the assay, between the LLOQ and upper limit of quantification (ULOQ), was defined as the lowest to the highest standard curve points achieving CV<20% and recovery 80-120% of expected. With these criteria, the linear range for the exemplary assay of Example 1 was 0.012 to 30 pg/mL. The calibration curves of the 5 separate runs aligned well (demonstrating a consistent signal with a wide dynamic range of 5-30,000 fg/mL) and demonstrate increasing signal across the entire range, but occasionally saturated at the top point, suggesting 0.005-10 pg/mL as a dynamic range for the exemplary assay of Example 1. The average, SD, CV and a signal to background ratio (S/B) for each dilution across the 5 separate runs shown in FIG. 6a are provided below in Table 2.

TABLE 2

| pg/mL | Avg | | CV | |
|---|---|---|---|---|
| calibrant | AEB | SD | (%) | S/B |
| 0 | 0.015 | 0.003 | 20.4 | 1 |
| 0.006 | 0.026 | 0.003 | 13.1 | 1.7 |
| 0.012 | 0.037 | 0.003 | 7.5 | 2.5 |
| 0.023 | 0.059 | 0.004 | 6.8 | 3.9 |
| 0.046 | 0.104 | 0.005 | 4.7 | 6.9 |
| 0.093 | 0.188 | 0.017 | 9.0 | 12.5 |
| 0.186 | 0.344 | 0.030 | 8.7 | 22.8 |
| 0.37 | 0.662 | 0.044 | 6.7 | 43.9 |
| 1.11 | 1.968 | 0.200 | 10.2 | 130.4 |
| 3.33 | 5.515 | 0.387 | 7.0 | 365.5 |
| 10 | 14.228 | 1.440 | 10.1 | 942.8 |
| 30 | 23.524 | 0.187 | 0.8 | 1558.7 |

LLOD = background + 2.5 sd = .015 + (2.5 × 0.003) = .0228 AEB, which calculates to a theoretical concentration of 0.002 pg/mL
LLOQ = first point on calibration curve showing CV <20% and Signal to Noise (S/N) >2 = 0.012 pg/mL

Dilution Linearity with Plasma

To assess dilution linearity and determine minimal required dilution (MRD) for testing serum and plasma samples, a set of 3 pooled serum samples from AD subjects having high tau levels and 1 pool plasma sample from AD subjects having high levels was titrated from 1:2 to 1:6 dilution (i.e., 1:2, 1:3, 1:4, 1:5 and 1:6 dilutions) in the sample diluent and measured according to the assay of Example 1. The amount of p217+tau detected from each dilution is then readjusted to provide an estimated concentration of p217+tau present in the undiluted serum or plasma sample and compared to an estimated concentration of p217+tau determined from a 1:3 dilution of the serum or plasma sample. The data obtained is shown as % of the estimated concentration of p217+tau in a 1:3 dilution of each of samples in FIG. 6b. Results obtained for each of the 3 serum samples are represented in FIG. 6b with a circle (e) symbol, a square (n) symbol and a triangle (A) symbol. Results obtained for the plasma sample is represented in FIG. 6b with an inverted triangle (V) symbol. As can be seen in FIG. 6b, each of the sample dilutions were within 20% of the other dilutions for all of the serum and plasma samples shown. This data demonstrates acceptable dilution linearity across the range of 1:2 to 1:6 dilution. Because serum and plasma samples contain low amounts of p217+tau, a dilution of 1:2 may be preferred.

### Example 9a: Technical Qualification of Assay for Detecting p217+tau in Plasma

### Precision

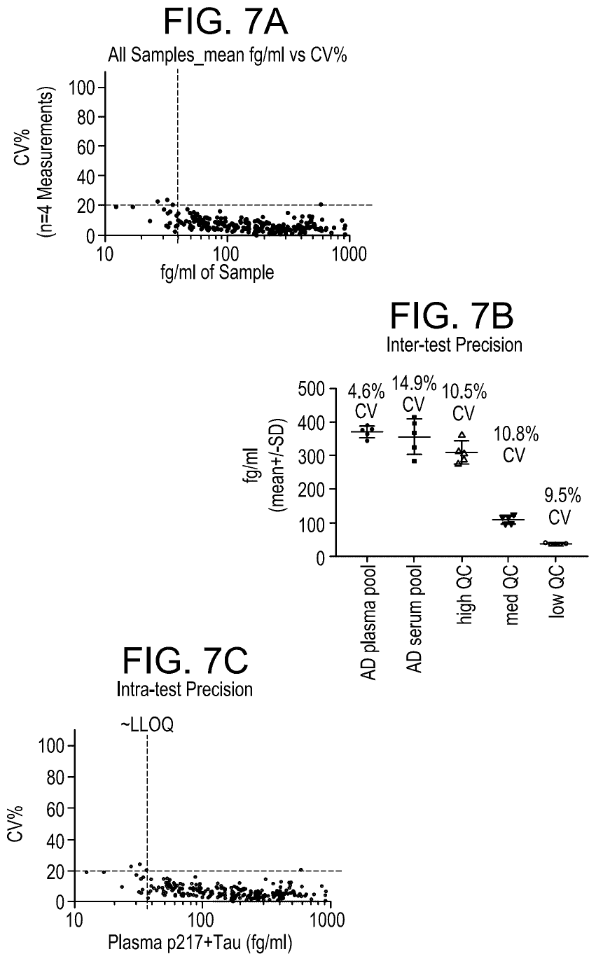

To assess inter-replicate precision of the p217+tau measurements from plasma, a cohort of 232 plasma samples (comprising both HV and AD subjects) was measured according to the exemplary assay of Example 1 in quadruplicate. Based on calibrant peptides standard curve, the LLOQ of the assay was determined to be 10 fg/mL. The LLOQ was adjusted to 20 fg/mL after accounting for 1:2 dilution of the samples. However, based on mapping the average amount of p217+tau detected (in fg/mL) from each sample to the % CV across each set of quadruplicate measurements for each sample, the data indicated that imprecision increased beginning below ˜40 fg/ml (FIG. 7a). The data of FIG. 7a shows that 93% of samples (216/232) measured above the LLOQ (concentration where CV<20%=40 fg/ml in this example, and shown as dotted lines) of the assay. In addition, all but 4 samples measured within the accepted limits of 20% CV for a Research Use Only (RUO) assay. The mean intra-test precision across all 232 plasma samples was 7.1% CV and the mean intra-test precision across those samples that are above the LLOQ was 6.7% CV.

To assess inter-test precision of the assay of Example 1, a panel of 3 quality control (QC) samples containing low, medium and high concentrations of the calibrant peptide in sample diluent were prepared along with a pooled sample of plasma from AD subjects having high tau levels and a pooled sample of serum from AD subjects having high tau levels. These samples were then measured according to the exemplary assay of Example 1 for 5 separate runs. The dilution corrected amounts of p217+tau detected from these samples are shown in FIG. 7b. For each sample, a mean value is shown as the longer horizontal line, with ±standard deviation (SD) of the dataset shown with the shorter lines above and below the mean value line. The inter-test precision was determined to be 5-15% CV for these 5 samples.

### Transferability Between Labs

To evaluate precision of the p217+tau assays between testing sites, a set of plasma samples obtained from HV and AD subjects may be tested, using the same lot of reagents at two separate locations. If the measurements for the samples are very similar between the two testing sites, then it is confirmed that the exemplary assay is transferable between labs.

### Analyte Stability

The stability of the endogenous p217+tau epitope in plasma may be assessed at various temperatures by aliquoting a pool of plasma obtained from AD subjects, and each aliquot subject to storage at 4° C., 22° C., or 37° C. for 1, 2, or 4 hrs. Also, a subset of aliquots may be freeze-thawed (−80° C. to 22° C.) 1, 2, 3 or 4 times. If no significant changes in signal are observed across these different storage conditions, then it would indicate that the tau species (and particular epitopes) recognized by the exemplary assay of Example 1 is sufficiently stable to allow for standard storage/testing procedures.

### Example 9b: Technical Qualification of Assay for Detecting p217+tau in Plasma

### Precision

Additionally, assay results for 227 plasma samples (157 mild-moderate, 70 cognitively normal subjects) from Example 9a were also analyzed to assess inter-replicate precision of the p217+tau measurements from plasma and shown in FIG. 7c. All samples shown in FIG. 7c were detected (presented signal>LLOD) and with acceptable precision (<25% CV, mean CV=7.1%). In fact, 223/227 samples (98.2%) presented with <20% CV. To better establish a lower limit of quantification (LLOQ), a cutoff of 37 fg/ml was set based on the point below which the plasma measurements were more likely to present >20% CV. As shown in FIG. 7c, 94.7% of all samples measured above this LLOQ.

### Example 10: Clinical Qualification of Assay for Detecting p217+tau in Plasma as Compared to CSF p217+tau and Tau PET

To assess the utility of the exemplary assay of Example 1 in diagnosis and staging of AD, three cohorts of plasma samples were obtained for p217+tau measurement using the assay. These measurements were analyzed for correlation with CSF p217+tau levels and/or TauPET SUVR, which are explained further below in the discussion of Cohort 3.

Cohort 1: Correlation of Plasma p217+tau to CSF p217+tau in AD Cohort

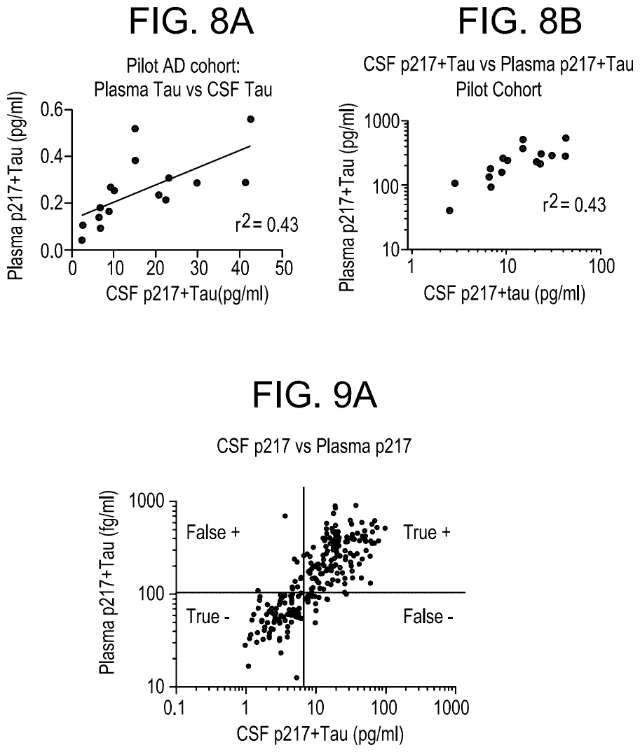

Cohort 1 evaluates correlation of p217+tau peptides detected in plasma to p217+tau detected in matching CSF from the same AD subjects. Lumbar Fluid (LF) CSF samples from each of 16 AD subjects (who were clinically diagnosed with mild-moderate dementia, with a Clinical Dementia Rating 1+) in a clinical study were measured using the pT3xhT43 assay described in Example 1 of the Kolb '492 patent. Plasma samples from the same 16 AD subjects were measured according to the exemplary assay of Example 1 described above. For each subject, the amount of p217+tau (in pg/mL) detected in the corresponding CSF sample (shown in the X-axis) is mapped to the amount of p217+tau detected in the corresponding plasma sample (shown in the Y-axis) in FIG. 8a and FIG. 8b, which shows the data of FIG. 8a in logarithmic scale. A linear regression line (R²=0.43, slope=0.007, p=0.006) along with the R² value for the linear regression line is shown in FIG. 8a. Plasma p217+tau concentrations were 1.95+/−0.23% (mean+/−SEM) of CSF p217+tau concentrations.

Cohort 2: Correlation of Plasma p217+tau to CSF p217+tau in Validation Cohort

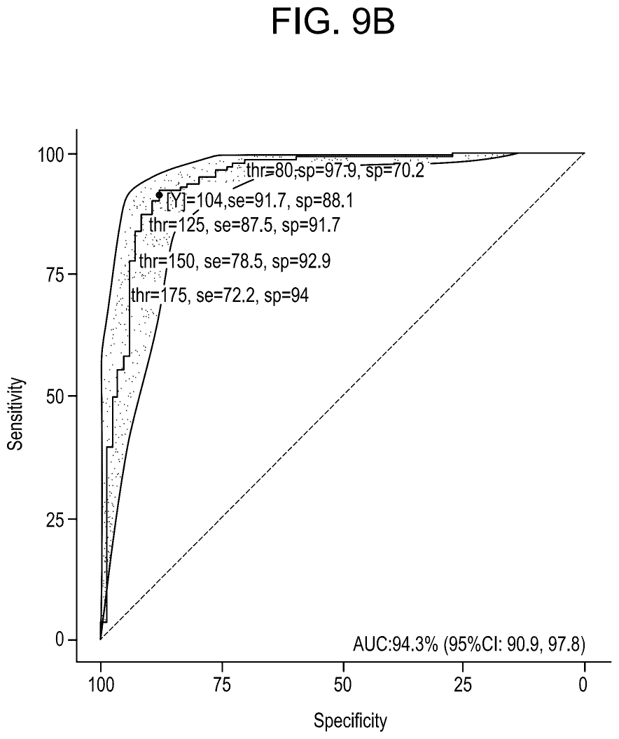

Cohort 2 evaluates correlation of p217+tau peptides detected in plasma to p217+tau detected in CSF in a larger cohort of subjects diagnosed with mild-moderate dementia (Clinical Dementia Rating 1+) in a clinical study (n=159) in addition to a cohort of asymptomatic subjects from another clinical study (n=70). LF CSF samples from Cohort 2, which comprises 229 subjects, were measured using the pT3xhT43 assay described in Example 1 of the Kolb '492 patent. Plasma samples from the same 229 subjects were measured according to the exemplary assay of Example 1. For each subject, the amount of p217+tau (in pg/mL) detected in the corresponding CSF sample (shown in the X-axis) is mapped to the amount of p217+tau (in fg/ml) detected in the corresponding plasma sample (shown in the Y-axis) in FIG. 9a. A linear regression (not shown) has R²=0.35, slope=6, and p<0.0001.

FIG. 9a also includes a vertical dotted line representing a first threshold value (6.6 μg/mL) above which the CSF samples would be indicative of a subject having or at risk of developing tauopathy (e.g., as demonstrated by an increased CSF p217+tau levels and/or increased TauPET SUVR, which are explained further below in the discussion of Cohort 3) and a corresponding horizontal dotted line representing a second threshold value (104 fg/mL) above which the plasma samples would be indicative of a subject having or at risk of developing tauopathy. The upper right quadrant labelled as “True+” corresponds to subjects where both CSF and plasma samples measured above the first and second threshold values, indicating that both CSF and plasma measurements concur in identifying the subject as having or at risk developing tauopathy. The lower left quadrant labelled as “True-” corresponds to subjects where both CSF and plasma samples measured below the first and second threshold values, indicating that both CSF and plasma measurements concur in identifying the subject as not being at risk of developing tauopathy. The upper left quadrant labelled as “False+” corresponds to subjects where CSF samples measured above the first threshold value, but the plasma samples measured below the second threshold value, indicating that the plasma measurements identify those subjects as having or being at risk of developing tauopathy while the CSF measurements do not. The lower right quadrant labelled as “False-” correspond to subjects where CSF samples measured below the first threshold value, but the plasma samples measured above the second threshold value, indicating that the CSF measurements identify those subjects as having or being at risk of developing tauopathy while the plasma measurements do not. The number of subjects identified in each quadrant of FIG. 9a are provided below in Table 3.

TABLE 3

| | True+ | True− | False+ | False− |
|---|---|---|---|---|
| # of subjects | 133 | 74 | 10 | 12 |
| % of subjects | 58 | 32 | 4 | 5 |

The data from FIG. 9a and Table 3 was used to create a Receiver-Operating Characteristic (ROC) curve, shown in FIG. 9b for the ability of the plasma measurements to differentiate those patients having CSF measurements that are above the second threshold value, indicating the subject as having or at risk of tauopathy, or below the second threshold value, indicating the subject as healthy or not being at risk of developing tauopathy. The plasma assay of Example 1 showed good specificity and sensitivity with AUC=0.943 (95% CI: 90.9, 97.8).

It was reported in the Kolb '492 patent that the “2-step” assay described therein was able to separate CSF samples from subjects having “biopsy+” brain biopsy samples from CSF samples from subjects having “biopsy-” brain biopsy samples, indicating that measurements of p217+tau in CSF may be indicative of a patient's clinical pathology for tauopathy, specifically, AD. In view of the data provided in FIGS. 9a and 9b and Table 3 herein, plasma measurements according to the assays and methods of the present application are also indicative of a patient's clinical pathology for tauopathy, corresponds to an increase in p217+tau in CSF, and useful as predictive biomarkers for detecting tauopathy in patients.

Cohort 3: Correlation of CSF p217+tau to PET Measurements of Tau

Cohort 3 evaluates correlation of p217+tau peptides detected in CSF to ¹⁸F-T807 (¹⁸F-AV-1451) tracer retention in brain tissue measured from PET images. The PET measurements of ¹⁸F-T807 tracer retention (Tau PET measurements) correspond to tau accumulation in brain tissue, which is a leading indicator for distinguishing subjects having or at risk of tauopathy from healthy subjects not at risk of developing tauopathy.

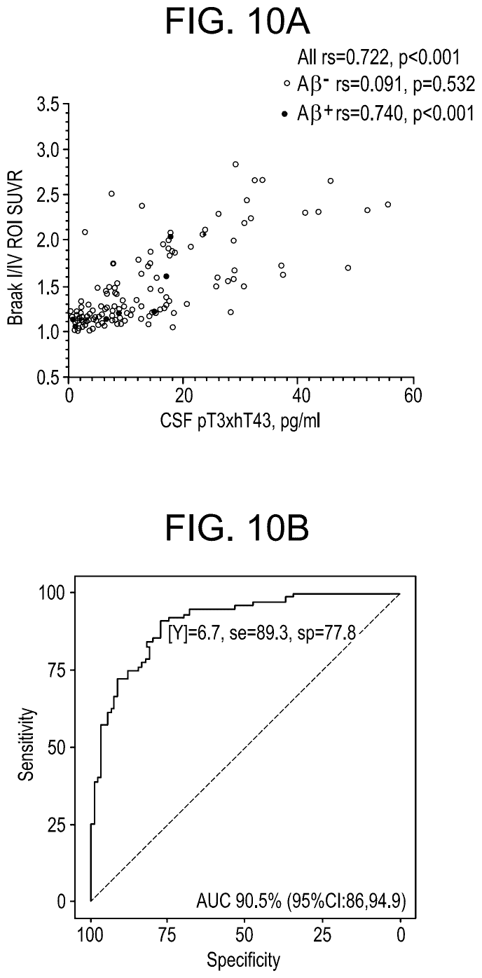

Cohort 3 includes 178 subjects in varying states of cognitive decline (Cognitively unimpaired controls, mild cognitive impairment, AD dementia and several other neurodegenerative disorders). Tau PET measurements were obtained from the Braak stage I-IV regions of interest (ROIs) of the brain of each of the subjects in Cohort 3. LF CSF samples collected simultaneously or concurrently from these subjects were measured using the pT3xhT43 assay described in Example 1 of the Kolb '492 patent. For each subject, the amount of p217+tau (in pg/mL) detected in the CSF sample (shown in the X-axis) is mapped to the standardized uptake value ratios (SUVRs) for the F¹⁸-labeled T807 tracer in the corresponding subject in FIG. 10a. The subjects of Cohort 3 demonstrated tau PET measurements having a range of SUVRs of the F¹⁸-labeled T807 tracer, as shown on the Y-axis of FIG. 10a. A linear regression (not shown) has R² 0.722, and p<0.0001.

The subjects of Cohort 3 can also be divided into two different subsets: those subjects that were found to correlate to an increase, or not, in amyloid-O (Aβ) deposition in brain tissue as measured by PET. Those having an increased amount of A3 are referred in Example 10 as Aβ+, while those that do not are referred to below as Aβ-. FIG. 10a shows those subjects that are Aβ+ in a darker shade, while the Aβ-subjects, most present in the lower left quadrant of the figure, are shown in a lighter shade. A linear regression (not shown) of p217+tau measurements in CSF to SUVRs of the F¹⁸-labeled T807 tracer for the Aβ+ subset has R²=0.740, and p<0.0001. A linear regression (not shown) of p217+tau measurements in CSF to SUVRs of the F¹⁸-labeled T807 tracer for the Aβ+ subset has R²=0.091, and p=0.532. The data from FIG. 10a was used to create a ROC curve for the ability of the CSF measurements to differentiate those patients having SUVRs of the F¹⁸-labeled T807 tracer above a threshold value (e.g., 1.25), indicating the subject as having or at risk of tauopathy, or below the threshold value, indicating the subject was healthy or not being at risk of developing tauopathy. The CSF measurements obtained using the pT3xhT43 assay described in Example 1 of the Kolb '492 patent showed good specificity and sensitivity for predicting this high T807 SUVR, with AUC=0.905 (95% CI: 86, 94.9) and also identified 6.6 μg/mL as a desired threshold value above which the CSF p217+tau measurements would be indicative of a subject having or at risk of developing tauopathy. This desired threshold value was thus used in the analysis of data obtained from Cohort 2, as discussed above, to analyze correlation between p217+tau measurements from plasma to TauPET. Therefore, the data provided in FIGS. 10a and 10b, when viewed in combination with the data obtained from Cohort 2, further demonstrates that plasma measurements according to the assays and methods of the present application are indicative of a patient's clinical pathology for tauopathy, corresponds to an increase in tau accumulation in brain tissue, and useful as predictive biomarkers for detecting tauopathy in patients.

### Example 11: Clinical Qualification of Assay for Detecting p217+tau in Plasma as Compared to CSF p217+tau and CSF p181tau

To assess the utility of the exemplary assay of Example 1 in diagnosis and staging of AD, plasma samples were obtained for p217+tau measurement using the assay. These measurements were analyzed for correlation with CSF p217+tau levels and/or CSF p181tau levels. The CSF p181tau level corresponds to an amount detected from CSF of a human tau protein or tau fragment that is phosphorylated at residue 181 of tau protein.

Correlation of Plasma p217+tau to CSF p217+tau in Validation Cohort

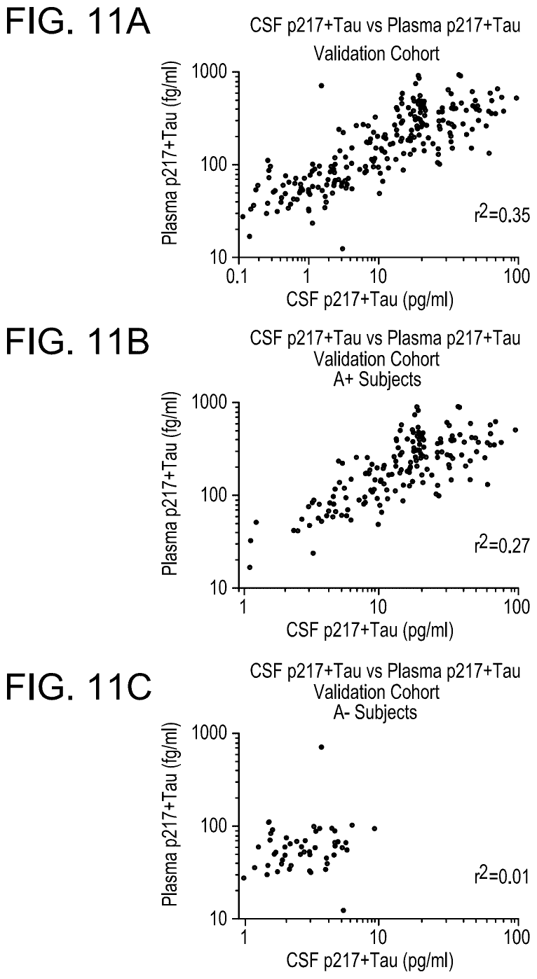

The same cohort used in Example 9b is used to evaluate correlation of p217+tau peptides detected in plasma to p217+tau detected in CSF. LF CSF samples from this cohort, which comprises 227 subjects, were measured using the pT3xhT43 assay described in Example 1 of the Kolb '492 patent. Plasma samples from the same 227 subjects were measured according to the exemplary assay of Example 1 described above. For each subject, the amount of p217+tau (in pg/mL) detected in the corresponding CSF sample (shown in the X-axis) is mapped to the amount of p217+tau (in fg/ml) detected in the corresponding plasma sample (shown in the Y-axis) in FIG. 11a. A linear regression (not shown) has R²=0.35. Plasma p217+tau concentrations were 1.87+/−0.11% (mean+/−SEM) of CSF p217+tau concentrations.

FIGS. 11b and 11c shows the data of FIG. 11a separated by amyloid status (e.g., A+ or A−) of the patients. A+ indicates patients who are considered amyloid positive, having a CSF Aβ42/40 ratio of <0.089. A− indicates patients who are considered amyloid negative, having a CSF Aβ42/40 ratio of >0.089. It is noted that 17 subjects from FIG. 11a are not included in either FIG. 11b or FIG. 11c because CSF amyloid data were not available for these patients. FIG. 11b shows data for the subset of patients that are A+(n=160) and FIG. 11c shows data for the subset of patients that are A− (n=50). A linear regression (not shown) for FIG. 11b has a R²=0.27. A linear regression (not shown) for FIG. 11c has a R²=0.01. Plasma p217+tau concentrations in A+ patients were 1.63+/−0.08% (mean+/−SEM) of CSF p217+tau concentrations. Plasma p217+tau concentrations in A− patients were 2.73+/−0.39% (mean+/−SEM) of CSF p217+tau concentrations. As shown in the data reported above, plasma to CSF p217+tau ratio was slightly, but significantly, lower in the amyloid positive cohort (p<0.0001 using unpaired t-test). Correlation of CSF p217+tau to CSF p181tau

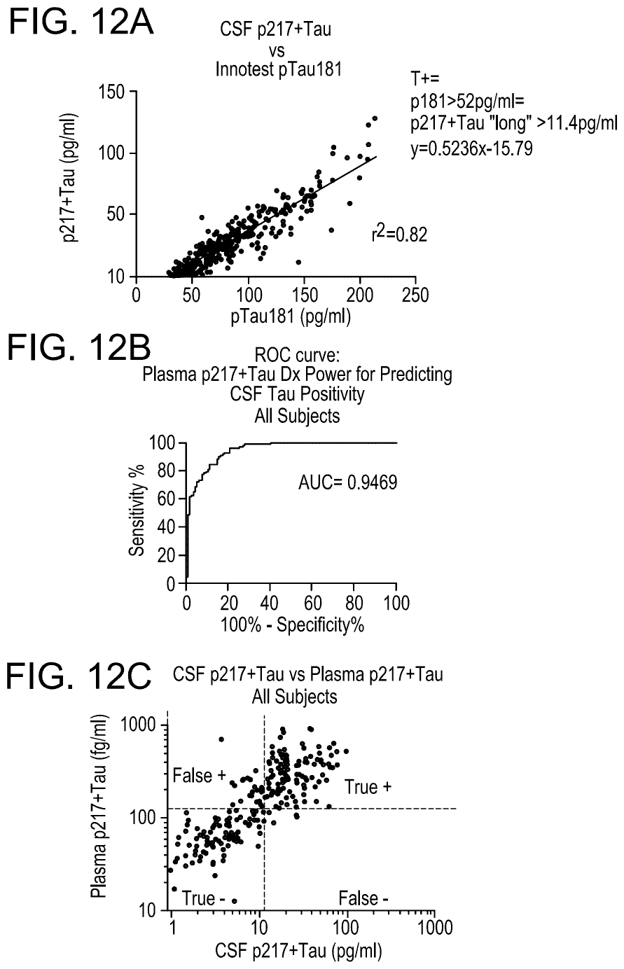

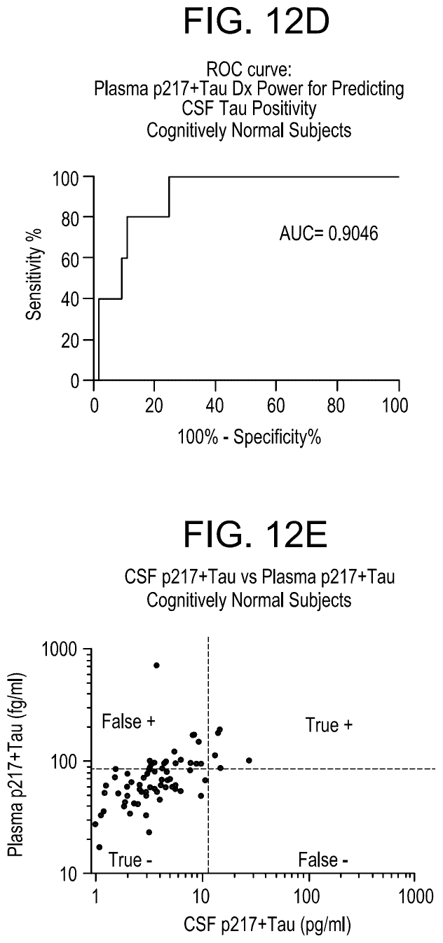

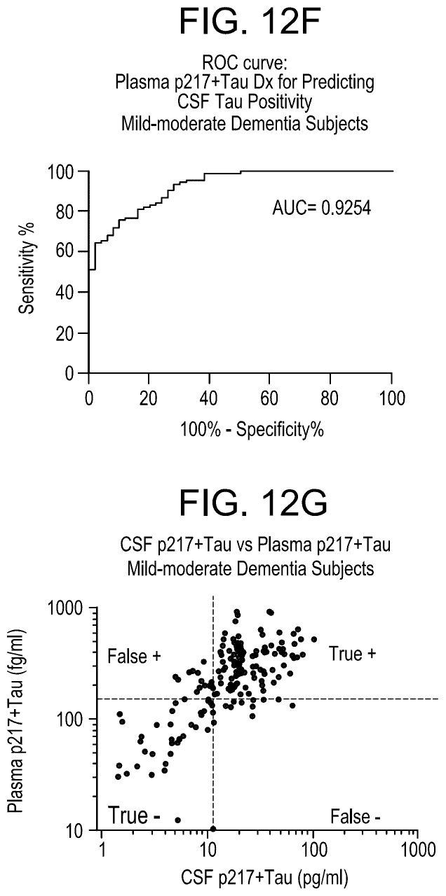

Correlation of p217+tau peptides detected in CSF to levels of p181 detected in CSF was evaluated. CSF samples from mild-moderate dementia subjects (n=286; 89% A+) were measured using the pT3xhT43 assay described in Example 1 of the Kolb '492 patent and by Innotest p181tau assay to determine concentrations of p217+tau and p181 detected from the CSF samples, respectively. For each subject, the amount of p181tau (in pg/mL) detected in the CSF sample (shown in the X-axis) is mapped to the amount of p217+tau (in pg/mL) detected in the CSF sample (shown in the Y-axis) in the corresponding subject in FIG. 12a. Those patients having an increased level of CSF p181 tau may be identified as T+, indicating that the patients have or at risk of tauopathy. Those patients having a level of CSF p181 below a certain threshold may be identified as T−, indicating that the patients are healthy or not being a risk of developing tauopathy. For this example, a CSF p181tau concentration of >52 μg/ml is identified as T+, while a CSF p181tau concentration of <52 μg/ml is referred to as T−. A linear regression of the data shown in FIG. 12a is used to correlate this threshold CSF p181tau concentration to a concentration of p217+tau in CSF. Based on this data, a CSF p181tau threshold value of 52 μg/ml correlates to 11.4 μg/ml of p217+tau in CSF. Therefore, a CSF p217+tau concentration of >11.4 μg/ml can be used to identify those patients that are T+, while a CSF p217+tau concentration of <11.4 μg/ml can be used to identify those patients that are T−.

The data from FIG. 11a was used to create a ROC curve (FIG. 12b) for the ability of the CSF p217+tau measurements to differentiate T+ from T− patients. The CSF measurements obtained using the pT3xhT43 assay described in Example 1 of the Kolb '492 patent showed high accuracy for predicting whether the patient would be T+ or T−, with AUC=0.9469. A Youden index analysis of the ROC curve determined a threshold value of plasma p217+tau for differentiating T+ from T− patients as 124.6 fg/ml.

The data from FIG. 11a is also shown in FIG. 12c with a vertical dotted line representing a first threshold value (as determined above to be 11.4 μg/ml), above which the CSF samples would be indicative of that of a T+ patient and a corresponding horizontal dotted line representing a second threshold value (as determined above to be 124.6 fg/ml), above which the plasma samples would be indicative of that of a T− patient. The upper right quadrant labelled as “True+” corresponds to subjects where both CSF and plasma samples measured above both the first and second threshold values, indicating that both CSF and plasma measurements concur in identifying the subject as having or at risk developing tauopathy. The lower left quadrant labelled as “True-” corresponds to subjects where both CSF and plasma samples measured below the first and second threshold values, indicating that both CSF and plasma measurements concur in identifying the subject as not being at risk of developing tauopathy. The upper left quadrant labelled as “False+” corresponds to subjects where CSF samples measured above the first threshold value, but the plasma samples measured below the second threshold value, indicating that the plasma measurements identify those subjects as having or being at risk of developing tauopathy while the CSF measurements do not. The lower right quadrant labelled as “False−” correspond to subjects where CSF samples measured below the first threshold value, but the plasma samples measured above the second threshold value, indicating that the CSF measurements identify those subjects as having or being at risk of developing tauopathy while the plasma measurements do not. FIG. 12c shows low false +/− rates at 10 and 2%, respectively. The number of subjects identified in each quadrant of FIG. 12c are provided below in Table 4.

TABLE 4

| | True+ | True− | False+ | False− |
|---|---|---|---|---|
| # of subjects | 108 | 91 | 24 | 4 |
| % of subjects | 48 | 40 | 10 | 2 |

FIGS. 12e and 12g shows the data of FIG. 12c separated by cognitively normal vs. mild-moderate dementia patients. FIG. 12e shows data for the cognitively normal subset of patients and FIG. 12g shows data for the mild-moderate dementia subset of patients.

The data from FIG. 12e was used to create a ROC curve (FIG. 12d) for the ability of the CSF p217+tau measurements to differentiate T+ from T− patients in a cognitively normal subset of patients (n=70). The CSF measurements for the cognitively normal subset showed a similar level of accuracy, with AUC=0.9045. A Youden index analysis of the ROC curve was used to determine a threshold value of plasma p217+tau for differentiating T+ from T− patients.

FIG. 12e also include a vertical dotted line representing a first threshold value (as determined above to be 11.4 μg/ml), above which the CSF samples would be indicative of that of a T+ patient and a corresponding horizontal dotted line representing a second threshold value (as determined by the ROC curve of FIG. 12d), above which the plasma samples would be indicative of that of a T− patient. The upper right quadrant labelled as “True+” corresponds to subjects where both CSF and plasma samples measured above both the first and second threshold values. The lower left quadrant labelled as “True-” corresponds to subjects where both CSF and plasma samples measured below the first and second threshold values. The upper left quadrant labelled as “False+” corresponds to subjects where CSF samples measured above the first threshold value, but the plasma samples measured below the second threshold value. The lower right quadrant labelled as “False-” correspond to subjects where CSF samples measured below the first threshold value, but the plasma samples measured above the second threshold value. FIG. 12e shows low false +/− rates at 23 and 0%, respectively. The number of subjects identified in each quadrant of FIG. 12e are provided below in Table 5.

TABLE 5

| | True+ | True− | False+ | False− |
|---|---|---|---|---|
| # of subjects | 5 | 49 | 16 | 0 |
| % of subjects | 7 | 70 | 23 | 0 |

The data from FIG. 12g was used to create a ROC curve (FIG. 12f) for the ability of the CSF p217+tau measurements to differentiate T+ from T− patients in a mild-moderate dementia subset of patients (n=157). The CSF measurements for the mild-moderate dementia subset showed a similar level of accuracy, with AUC=0.9254. A Youden index analysis of the ROC curve was used to determine a threshold value of plasma p217+tau for differentiating T+ from T− patients.

FIG. 12g also shows a vertical dotted line representing a first threshold value (as determined above to be 11.4 μg/ml), above which the CSF samples would be indicative of that of a T+ patient and a corresponding horizontal dotted line representing a second threshold value (as determined by the ROC curve of FIG. 12f), above which the plasma samples would be indicative of that of a T− patient. The upper right quadrant labelled as “True+” corresponds to subjects where both CSF and plasma samples measured above both the first and second threshold values. The lower left quadrant labelled as “True-” corresponds to subjects where both CSF and plasma samples measured below the first and second threshold values. The upper left quadrant labelled as “False+” corresponds to subjects where CSF samples measured above the first threshold value, but the plasma samples measured below the second threshold value. The lower right quadrant labelled as “False-” correspond to subjects where CSF samples measured below the first threshold value, but the plasma samples measured above the second threshold value. FIG. 12g shows low false +/− rates at 9 and 4%, respectively. The number of subjects identified in each quadrant of FIG. 12g are provided below in Table 6.

TABLE 6

| | True+ | True− | False+ | False− |
|---|---|---|---|---|
| # of subjects | 100 | 36 | 14 | 7 |
| % of subjects | 64 | 23 | 9 | 4 |

The data shown in FIGS. 12e and 12g show that plasma measurements according to the assays and methods of the present application provide similar predictive power in each of cognitively normal and mild-moderate dementia subsets.

### Example 12: Clinical Qualification of Assay for Detecting p217+tau in Plasma as Compared to CSF β-Amyloid

To assess the utility of the exemplary assay of Example 1 in diagnosis and staging of AD, plasma samples were obtained for p217+tau measurement using the assay. These measurements were analyzed for correlation with CSF β-amyloid levels.

Correlation of Plasma p217+tau to CSF β-Amyloid Levels

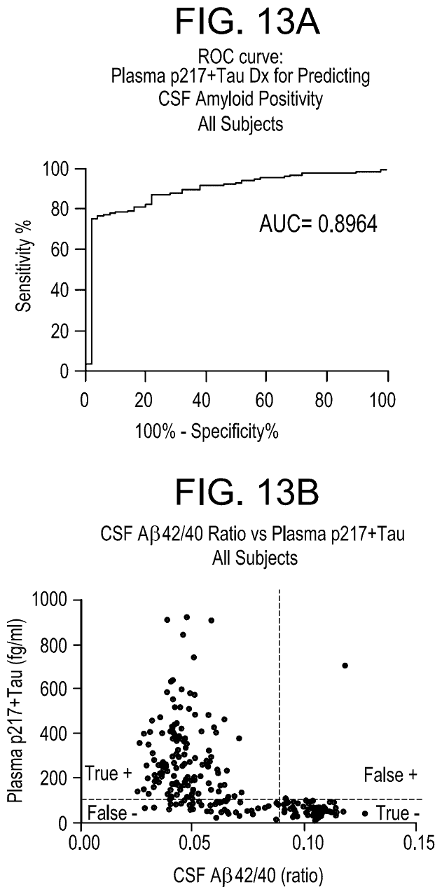

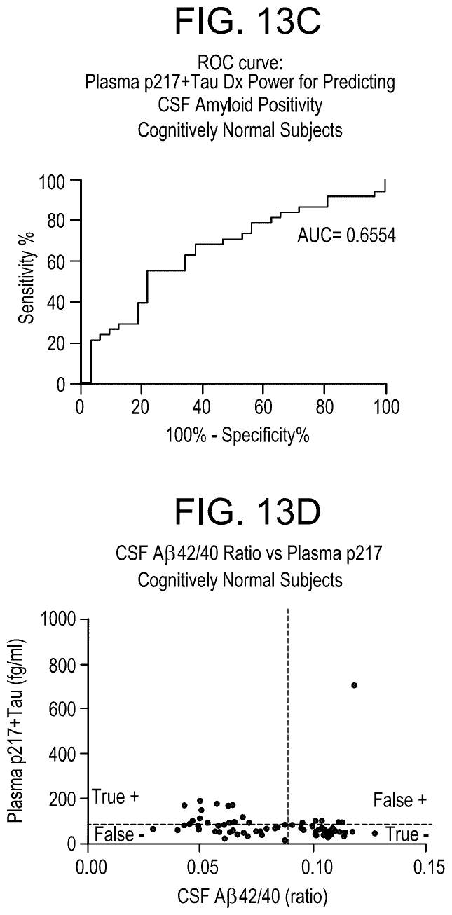

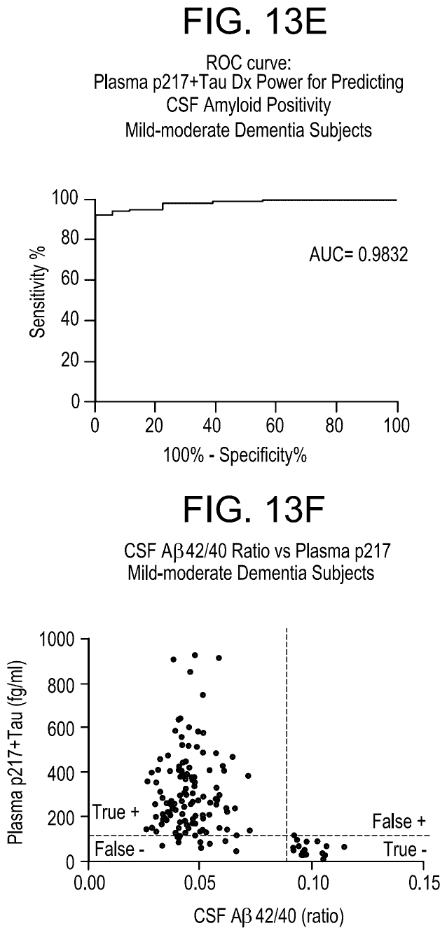

A cohort of 210 patients was used to evaluate correlation of p217+tau peptides detected in plasma to CSF β-amyloid levels. CSF samples from this cohort were measured to determine an amount of Aβ42 and an amount of Aβ40 present in the samples. Plasma samples from the same 227 subjects were measured according to the exemplary assay of Example 1 described above. A ratio of an amount of Aβ42 detected to an amount of Aβ40 is a leading indicator for distinguishing subjects having or at risk of amyloidogenic disease from healthy subjects not at risk of developing amyloidogenic disease. For each subject, a ratio of an amount of Aβ42 detected to an amount of Aβ40 (Aβ42/40 ratio) in the corresponding CSF sample (shown in the X-axis) is mapped to the amount of p217+tau (in fg/ml) detected in the corresponding plasma sample (shown in the Y-axis) in FIG. 13a. Those patients having a decreased Aβ42/40 ratio may be identified as A+, indicating that the patients having or at risk of amyloidogenic disease. Those patients having an increased Aβ42/40 ratio may be identified as A−, indicating that the patients are healthy or not being a risk of developing amyloidogenic disease. For this example, a Aβ42/40 ratio of <0.089 is identified as A+, while a Aβ42/40 ratio of >0.089 is referred to as A−.

The data from FIG. 13b was used to create a ROC curve (FIG. 13a) for the ability of the plasma p217+tau measurements to differentiate patients having T+ from T− patients. The plasma p217+tau showed high accuracy for predicting whether the patient would be A+ or A−, with AUC=0.8964. A Youden index analysis of the ROC curve determined a threshold value of plasma p217+tau for differentiating A+ from A− patients as 103.9 fg/ml.

FIG. 13b also includes a vertical dotted line representing a first threshold value (as determined above as 0.089), below which Aβ42/40 ratio would be indicative of that of a A+ patient and a corresponding horizontal dotted line representing a second threshold value (as determined above to be 103.9 fg/ml), below which the plasma p217+tau levels would be indicative of that of a A− patient. The upper left quadrant labelled as “True+” where the Aβ42/40 ratio is below the first threshold value and the plasma p217+tau levels is above the second threshold value, indicating that both the Aβ42/40 ratio and plasma p217+tau measurement concur in identify the subject as having or at risk of developing amyloidogenic disease. The lower right quadrant labelled as “True-” corresponds to subjects where both the Aβ42/40 ratio is above the first threshold value and the plasma p217+tau levels is below the second threshold value, indicating that both Aβ42/40 ratio and the plasma p217+tau measurement concur in identifying the subject as not being at risk of developing amyloidogenic disease. The upper right quadrant labelled as “False+” corresponds to subjects where the Aβ42/40 ratio and the plasma p217+tau levels are above the first and second threshold values, respectively, indicating that the plasma p217+tau levels identify those subjects as having or being at risk of developing amyloidogenic disease while the Aβ42/40 ratio do not. The lower left quadrant labelled as “False-” correspond to subjects where the Aβ42/40 ratio is below the first threshold value and the plasma p217+tau levels measured below the second threshold value, indicating that the Aβ42/40 ratio identify those subjects as having or being at risk of developing amyloidogenic disease while the plasma p217+tau levels do not. FIG. 13b shows low false +/− rates at 1 and 18%, respectively. The number of subjects identified in each quadrant of FIG. 13b are provided below in Table 7.

TABLE 7

| | True+ | True− | False+ | False− |
|---|---|---|---|---|
| # of subjects | 123 | 48 | 2 | 37 |
| % of subjects | 58 | 23 | 1 | 18 |

FIGS. 13d and 13f shows the data of FIG. 13b separated by cognitively normal vs. mild-moderate dementia patients. FIG. 13d shows data for the cognitively normal subset of patients and FIG. 13f shows data for the mild-moderate dementia subset of patients.

The data from FIG. 13d was used to create a ROC curve (FIG. 13c) for the ability of the plasma p217+tau measurements to differentiate patients having A+ from A− patients in the cognitively normal subset of patients (n=70). The plasma p217+tau measurements for the cognitively normal subset of patients has a ROC curve having a AUC=0.6554. A Youden index analysis of the ROC curve was used to determine a threshold value of plasma p217+tau for differentiating A+ from A− patients.

FIG. 13d also includes a vertical dotted line representing a first threshold value (as determined above as 0.089), below which Aβ42/40 ratio would be indicative of that of a A+ patient and a corresponding horizontal dotted line representing a second threshold value (as determined by the ROC curve of FIG. 13c), below which the plasma p217+tau levels would be indicative of that of a A− patient. The upper left quadrant labelled as “True+” where the Aβ42/40 ratio is below the first threshold value and the plasma p217+tau levels is above the second threshold value. The lower right quadrant labelled as “True-” corresponds to subjects where both the Aβ42/40 ratio is above the first threshold value and the plasma p217+tau levels is below the second threshold value. The upper right quadrant labelled as “False+” corresponds to subjects where the Aβ42/40 ratio and the plasma p217+tau levels are above the first and second threshold values, respectively. The lower left quadrant labelled as “False-” correspond to subjects where the Aβ42/40 ratio is below the first threshold value and the plasma p217+tau levels measured below the second threshold value. FIG. 13d shows false +/− rates at 10 and 24%, respectively. The number of subjects identified in each quadrant of FIG. 13d are provided below in Table 8.

TABLE 8

| | True+ | True− | False+ | False− |
|---|---|---|---|---|
| # of subjects | 21 | 25 | 7 | 17 |
| % of subjects | 30 | 36 | 10 | 24 |

The data from FIG. 13f was used to create a ROC curve (FIG. 13e) for the ability of the plasma p217+tau measurements to differentiate patients having A+ from A− patients in the mild-moderate dementia subset of patients (n=140). The plasma p217+tau measurements for provided high accuracy in the mild-moderate dementia subset of patients with an AUC=0.9832. A Youden index analysis of the ROC curve was used to determine a threshold value of plasma p217+tau for differentiating A+ from A− patients.

FIG. 13f also includes a vertical dotted line representing a first threshold value (as determined above as 0.089), below which Aβ42/40 ratio would be indicative of that of a A+ patient and a corresponding horizontal dotted line representing a second threshold value (as determined by the ROC curve of FIG. 13e), below which the plasma p217+tau levels would be indicative of that of a A− patient. The upper left quadrant labelled as “True+” where the Aβ42/40 ratio is below the first threshold value and the plasma p217+tau levels is above the second threshold value. The lower right quadrant labelled as “True-” corresponds to subjects where both the Aβ42/40 ratio is above the first threshold value and the plasma p217+tau levels is below the second threshold value. The upper right quadrant labelled as “False+” corresponds to subjects where the Aβ42/40 ratio and the plasma p217+tau levels are above the first and second threshold values, respectively. The lower left quadrant labelled as “False-” correspond to subjects where the Aβ42/40 ratio is below the first threshold value and the plasma p217+tau levels measured below the second threshold value. FIG. 13f shows false +/− rates at 0 and 6%, respectively. The number of subjects identified in each quadrant of FIG. 13f are provided below in Table 9.

TABLE 9

| | True+ | True− | False+ | False− |
|---|---|---|---|---|
| # of subjects | 113 | 18 | 0 | 9 |
| % of subjects | 81 | 13 | 0 | 6 |

The data provided in FIGS. 13d and 13f show that plasma measurements according to the assays and methods of the present application provide improvement in predictive accuracy in the mild-moderate dementia subset of patients.

### Example 13: Correlation of Plasma p217+tau to CSF p217+tau with Biochemical Purification

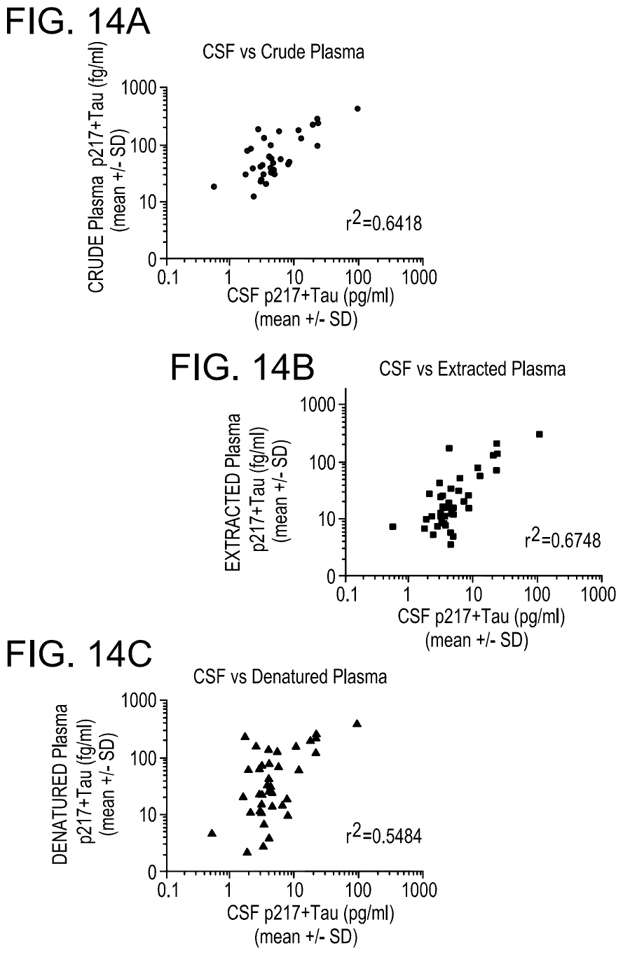

An additional cohort of 36 patients were tested to further evaluate correlation of p217+tau peptides detected in plasma to p217+tau detected in CSF. CSF samples in this cohort were measured using the pT3xhT43 assay described in Example 1 of the Kolb '492 patent. Plasma samples from the same subjects were measured in three different ways. First, crude plasma samples are measured according to the exemplary assay of Example 1. Second, tau peptides are chemically extracted from plasma samples and measured according to the exemplary assay of Example 1. Third, plasma samples are semi-denatured according to Example 2 so that interfering proteins are denatured by heat. For each subject, the amount of p217+tau (in pg/mL) detected in the corresponding CSF sample (shown in the X-axis) is mapped to the amount of p217+tau (in fg/ml) detected in the corresponding crude plasma sample (shown in the Y-axis) in FIG. 14a. A linear regression (not shown) for FIG. 14a has a R²=0.6418. The amount of p217+tau (in pg/mL) CSF sample (shown in the X-axis) is mapped to the amount of p217+tau (in fg/ml) detected in the corresponding chemically extracted plasma sample (shown in the Y-axis) in FIG. 14b. A linear regression (not shown) for FIG. 14b has a R²=0.6748. The amount of p217+tau (in pg/mL) CSF sample (shown in the X-axis) is mapped to the amount of p217+tau (in fg/ml) detected in the corresponding semi-denatured plasma sample (shown in the Y-axis) in FIG. 14c. A linear regression (not shown) for FIG. 14c has a R²=0.5484.

### Example 14: Quantification of Total p217+tau in Subjects Treated with Exogenous Anti-Tau Antibodies

The assays and methods of the present application may be used to detect total p217+tau in plasma samples obtained from subjects undergoing treatment with an anti-tau antibody, in particular, an anti-p217+tau antibody. However, detection of p217+tau in plasma samples of subject undergoing treatment with an anti-tau antibody may suffer from interferences and/or artifacts caused by the presence of the treatment antibody in the plasma samples. Therefore, the steps for generating a semi-denatured sample fluid described in Example 2 may be used to treat a plasma sample from subjects undergoing treatment with an anti-tau antibody, to reduce interference from the treatment antibody while allowing the p217+tau signal to remain in the semi-denatured sample fluid.

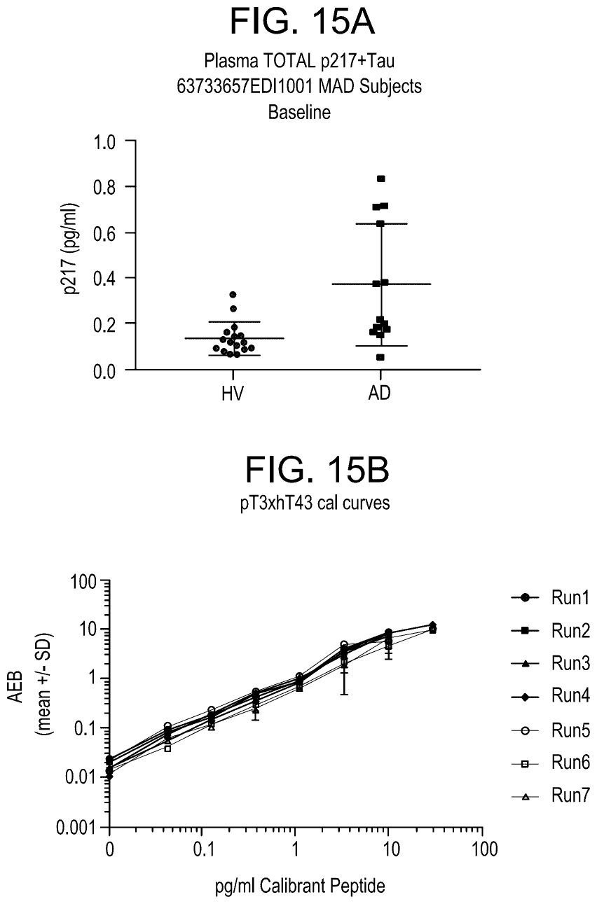

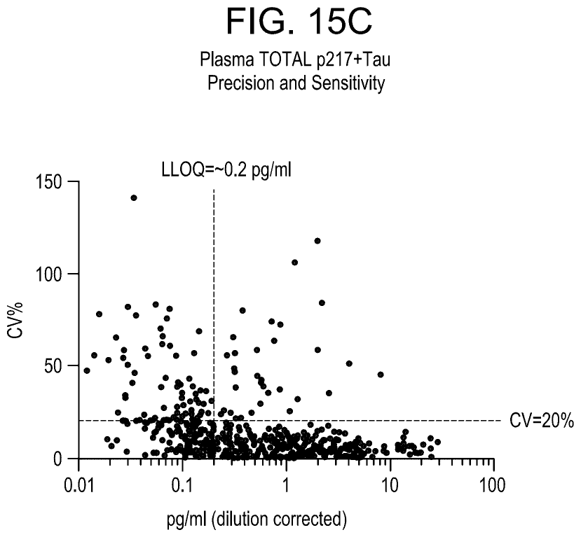

Example 14 modifies the exemplary assay of Example 1 with steps for denaturing the samples in the same manner as those described for obtaining semi-denatured sample fluids according to Example 2. The modified exemplary assay is evaluated using plasma samples of HV and AD subjects. These plasma samples were heated and measured according to the exemplary assay of Example 10 and the results (in pg/mL of p217+tau detected) are shown in FIG. 15a. The left side of FIG. 15a shows data corresponding to HV subjects and the right side of the graph shows data corresponding to AD subjects. For each category of subjects, a mean value is shown as the longer horizontal line, with ±standard deviation (SD) of the dataset shown with the shorter lines above and below the mean value line. As can be seen in FIG. 15a, semi-denatured plasma samples of AD subjects measured significantly higher than those obtained from HV subjects, mirroring results seen in crude plasma samples with the Example 1 assay.

The modified exemplary assay of Example 14 was used to measure a panel of 585 plasma samples from a Phase 1 clinical trial of an anti-p217+tau antibody therapy to study to evaluate sensitivity and precision. Representative standard curves were generated from 8 separate runs of semi-denatured sample fluids obtained from different dilutions of the calibrant peptides as specified in Example 1 and are shown in FIG. 15b. The linear range, between the LLOQ and ULOQ, for assaying semi-denatured samples as described in Example 14 was defined as the lowest to the highest standard curve points achieving CV<20% and recovery 80-120% of expected, and then correction for the 1:6 dilution of the samples. With these criteria, the linear range for the modified exemplary assay of Example 10 was ˜0.24 to 180 pg/mL. However to assess precision of the modified exemplary assay of Example 14 with actual samples, the average amount of p217+tau detected (in fg/mL) from each semi-denatured plasma sample was mapped to the % CV for each sample (FIG. 15c). The data of FIG. 15c shows that CV range from 0-141% with a mean of 14%. In addition, 82% of the samples shown in FIG. 15c have a CV<20% and that 66% of the samples are within linear range. The vertical dashed line represents the concentration in semi-denatured samples where imprecision appears to increase, and thus is the sample-defined LLOQ of ˜0.2 μg/ml with the method of Example 14.

In view of this data, it is further contemplated that the assays and methods of the present application may be combined with preanalytical manipulation of plasma samples to measure the levels of p217+tau in plasma samples obtained from subjects having exogenously administered anti-tau antibodies to monitor pharmacological effects of anti-tau antibody therapy on p217+tau levels in plasma.

### Example 15: Computer-Implemented Method of Detecting and/or Predicting Tauopathy

Example 15 describes an exemplary computer-implemented method for analyzing blood-based measurements of biomarkers for tauopathy to improve detection and/or prediction of tauopathy in a subject. In particular, one of the biomarker measurements used in Example 15 is p217+tau levels assayed from serum samples. However, it is contemplated that the method described in Example 15 is equally applicable to p217+tau levels measured in plasma, such as those measurements obtained using the exemplary assay of Example 1.

In Example 15, a panel of 23 blood-based biomarkers were assayed in plasma and serums samples from 199 subjects having mild to moderate AD in a Phase III clinical study. The 23 blood-based biomarkers included p217+tau, NFL, adiponectin, leptin, and other inflammatory and metabolic markers. In addition, for each of these patients, p217+tau levels were also measured in CSF using the pT3xhT43 assay described in Example 1 of the Kolb '492 patent. A subject was determined to be “T-positive” if the amount of p217+tau peptides measured from CSF of the subject exceeded 21 pg/mL, this concentration corresponds to a commonly used cutoff of 70 μg/mL with Innotest p¹⁸1-tau for defining “T” status. Data corresponding to the 23 biomarker measurements and p217+tau measurements in CSF, along with patient demographic data (e.g., age and sex) for all 199 subjects were then separated into two different data sets: a training set (n=150) and a holdout set (n=49). The training set was analyzed to select features that had higher correlation to an increased level of CSF—those subjects that have been identified as “T-positive.” The selected features include blood-based measurements for p217+tau, NFL, adiponectin and leptin. A plurality of machine learning modules was trained using the training set. Specifically, a support vector machine module, a random forest module, a logistic regression module, a gradient boosting module were trained using the training set. An ensemble of all of these trained machine learning modules was used to generate an outcome.

To evaluate the sensitivity and accuracy of the outcomes generated by the ensemble machine learning module, data from the holdout set was analyzed by the ensemble machine learning module to generate a determination of whether data for each subject of the holdout set corresponds to a “T-positive” subject. The determinations generated by the ensemble machine learning module are then compared to the actual “T-positive” status of the subjects of the holdout set to assess sensitivity and accuracy of the ensemble machine learning module. The ensemble machine learning module was evaluated in this manner using different subsets of biomarkers as shown below in Table 10.

TABLE 10

| Data subsets | Biomarkers Features |
|---|---|
| Control | No biomarker, analyze using age and sex |
| | of subject |
| 1 | Serum p217 + tau |
| 2 | Serum p217 + tau and NFL |
| 3 | Serum p217 + tau, NFL, and adiponectin |
| 4 | Serum p217 + tau, NFL, adiponectin, and |
| | leptin |
| 5 | NFL, adiponectin, and leptin |

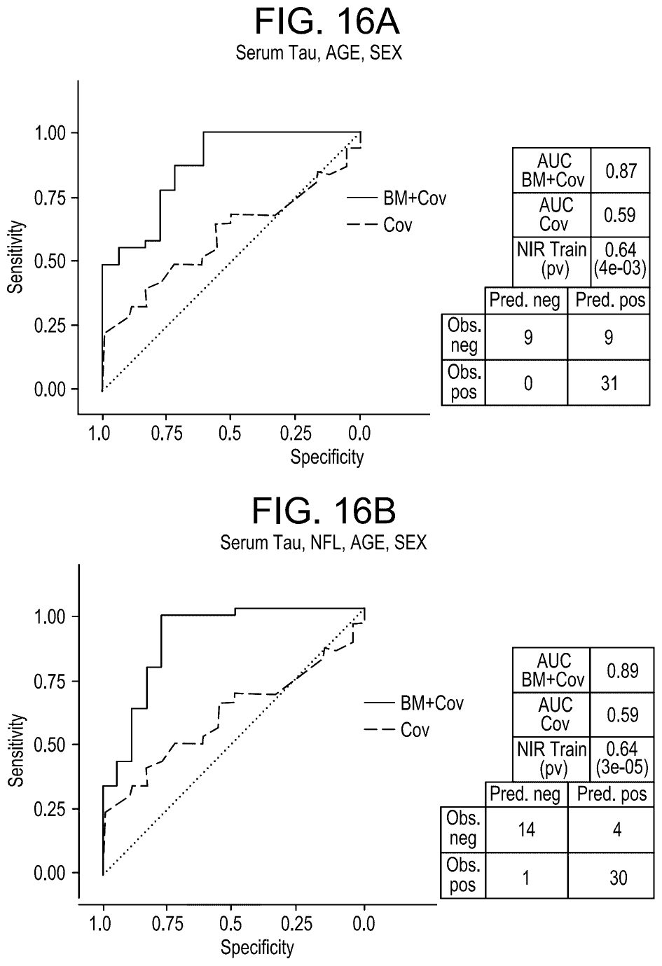

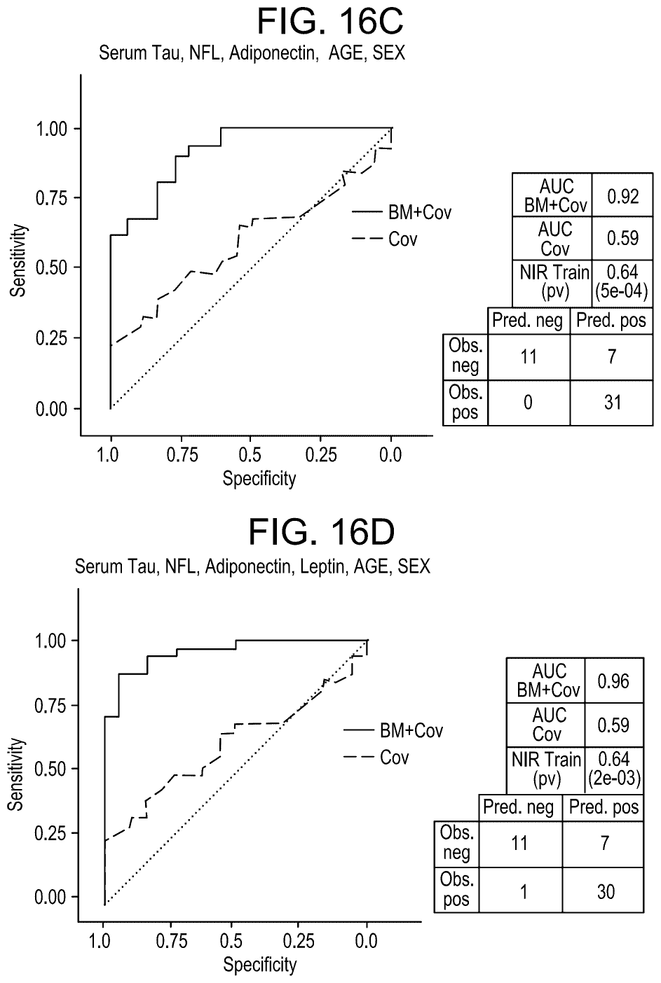

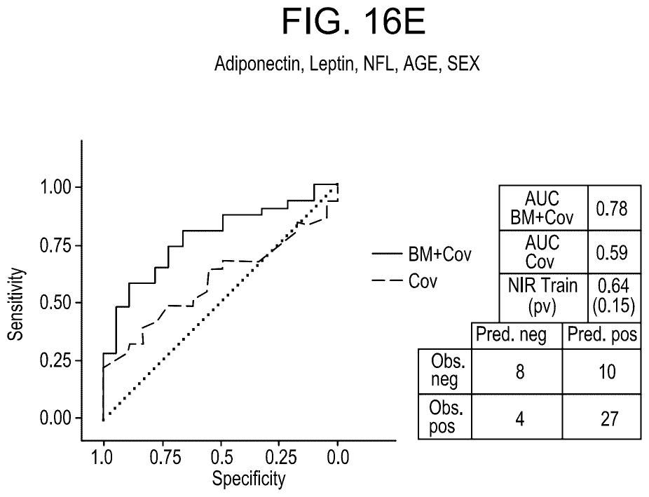

The control data subset includes data from the holdout set, excluding any biomarker data. Specifically, the control data subset was analyzed by the ensemble machine learning module using non-biomarker features—age and sex—of each subject. Analysis generated by the ensemble machine learning module for the control data subset was used to create a ROC curve for the ability of the ensemble machine learning module to differentiate the “T-positive” status of the subjects in the hold outset without any biomarkers, which is shown as dashed lines in FIGS. 16a-16e. The AUC of the ROC curve for the ensemble machine learning module analyzing the control data subset, without any biomarker data, is 0.59.

Each of Data Subsets 1-5 includes the control data subset and data from the holdout set corresponding to the biomarkers specified above in Table 10. Analyses generated by the ensemble machine learning module for each of Data Subsets 1-5 are provided below in Tables 5-9. Subjects having p217+tau measurements in CSF in excess of 21 pg/mL are listed as “Observed+” while those below are listed as “Observed-” in Tables 5-9 below. Subjects identified by the ensemble machine learning module as corresponding to “T-positive” status are listed as “Predicted+” while those not identified as corresponding to “T-positive” status are listed as “Predicted-” in Tables 5-9.

Results from analyses of Data Subset 1 utilizing p217+tau measurements from serum as a feature is provided in Table 11. Data from Table 11 was used to create a ROC curve for the ability of the ensemble machine learning module to differentiate the “T-positive” status of the subjects in the hold outset with p217+tau measurements from serum, which is shown as a solid line in FIG. 16a. The AUC of the ROC curve is 0.87.

TABLE 11

| | Predicted+ | Predicted− |
|---|---|---|
| Observed+ | 9 | 9 |
| Observed− | 0 | 31 |

Results from analyses of Data Subset 2, having p217+tau measurements from serum and data for NFL are features, is provided in Table 12. Data from Table 12 was used to create a ROC curve for the ability of the ensemble machine learning module to differentiate the “T-positive” status of the subjects in the hold outset with p217+tau measurements from serum and data for NFL, which is shown as a solid line in FIG. 16b. The AUC of the ROC curve is 0.89.

TABLE 12

| | Predicted+ | Predicted− |
|---|---|---|
| Observed+ | 14 | 4 |
| Observed− | 1 | 30 |

Results from analyses of Data Subset 3, having p217+tau measurements from serum, and data for NFL and adiponectin as features, is provided in Table 13. Data from Table 13 was used to create a ROC curve for the ability of the ensemble machine learning module to differentiate the “T-positive” status of the subjects in the hold outset with p217+tau measurements from serum, and data for NFL and adiponectin, which is shown as a solid line in FIG. 16c. The AUC of the ROC curve is 0.92.

TABLE 13

| | Predicted+ | Predicted− |
|---|---|---|
| Observed+ | 11 | 7 |
| Observed− | 0 | 31 |

Results from analyses of Data Subset 4, having p217+tau measurements from serum, and data for NFL, adiponectin and leptin as features, is provided in Table 14. Data from Table 14 was used to create a ROC curve for the ability of the ensemble machine learning module to differentiate the “T-positive” status of the subjects in the hold outset with p217+tau measurements from serum, and data for NFL, adiponectin and leptin, which is shown as a solid line in FIG. 16d. The AUC of the ROC curve is 0.96.

TABLE 14

| | Predicted+ | Predicted− |
|---|---|---|
| Observed+ | 11 | 7 |
| Observed− | 1 | 30 |

Results from analyses of Data Subset 5, having data for NFL, adiponectin and leptin as features, is provided in Table 15. Data from Table 15 was used to create a ROC curve for the ability of the ensemble machine learning module to differentiate the “T-positive” status of the subjects in the hold outset with data for NFL, adiponectin and leptin but not p217+tau measurements from serum, which is shown as a solid line in FIG. 16e. The AUC of the ROC curve is 0.78.

TABLE 15

| | Predicted+ | Predicted− |
|---|---|---|
| Observed+ | 8 | 10 |
| Observed− | 4 | 27 |

The biomarker feature set used in Example 15 consisted of serum p217+tau, NFL, adiponectin, and leptin, each of which has a Spearman correlation to CSF p127+tau levels of 0.47, 0.37, 0.16, −0.23, respectively. Machine learning analysis using serum p217+tau, age and sex as features resulted in improvement performance, having an AUC of 0.87, compared to 0.59 for the control data subset. Increasing complexity to the machine learning analysis by sequentially adding NFL, adiponectin and leptin as features progressively improved performance, resulting in AUCs of 0.89, 0.92, and 0.96, respectively. When the machine learning analysis includes all 4 biomarkers (p217+tau measurements from serum, and data for NFL, adiponectin and leptin) as features, accuracy was at 0.84, significantly higher than the no information rate (p<0.005). Removing p217+tau measurements from serum as a feature reduced the AUC to 0.78 and therefore suggests that p217+tau measurements would be a significant biomarker for predicting “T-positive” status.

With all 4 biomarkers, accuracy was at 0.84, significantly higher than the no information rate (p<0.005). Omitting serum Tau from the full model reduced the AUC to 0.78.

In summary, blood-based biomarkers can be used to identify Tau positive subjects. Serum p217+tau was the best single analyte for predicting tauopathy or brain pathology of tauopathy (e.g., amount of p217+tau detected in CSF) in mild to moderate AD subjects.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. An assay method of detecting p217+tau peptides in a subject, the method comprising:
obtaining a plasma sample from the subject;
contacting the plasma sample with a capture antibody directed against a p217+tau epitope to bind the capture antibody to p217+tau peptides in the plasma sample to form antibody-peptide complexes;
washing the antibody-peptide complexes;
contacting the antibody-peptide complexes with a detection antibody to bind the detection antibody to the antibody-peptide complexes; and
detecting the detection antibody to determine an amount of the p217+tau peptides in the plasma sample.

2. The method of claim 1, wherein the capture antibody is immobilized on a solid phase.

3. The method of claim 2, wherein the solid phase is a magnetic bead.

4. The method of claim 1, wherein the capture antibody binds to an epitope containing amino acids 210-220 of human tau protein.

5. The method of claim 1, wherein the detection antibody binds to an epitope comprising amino acids 7-20 or 116-127 of human tau protein.

6. The method of claim 4, wherein the capture antibody is pT3.

7. The method of claim 5, wherein the detection antibody is pT82.

8. The method of claim 3, wherein the plasma sample is diluted with a sample diluent before contacting with the capture antibody, the sample diluent comprising at least one of a non-ionic surfactant and tris(hydroxymethyl)aminomethane.

9. A method of detecting tauopathy in a subject, the method comprising:
obtaining a plasma sample from the subject;
detecting an amount of p217+tau peptides in the plasma sample using an assay, wherein the assay uses a capture antibody directed against a p217+tau epitope to bind the capture antibody to p217+tau peptides in the plasma sample to form antibody-peptide complexes and a detection antibody to bind the detection antibody to the antibody-peptide complexes; and
determining the subject as having tauopathy or is at risk of developing tauopathy when the amount of the p217+tau peptides is above a predetermined threshold value, wherein the predetermined threshold value is above a Lower Limit of Quantification (LLOQ) of the assay.

10. The method of claim 9, wherein the assay does not concentrate the p217+tau peptides from the plasma sample by immunoprecipitation before measuring the amount of the p217+tau peptides present.

11. The method of claim 9, wherein the plasma sample is crude plasma.

12. The method of claim 9, wherein the LLOQ corresponds to a 15-25% coefficient of variation (CV) of the assay.

13. The method of claim 12, wherein the LLOQ corresponds to a 20% CV of the assay.

14. The method of claim 9, wherein the predetermined threshold value is at least 3 times of the LLOQ.

15. The method of claim 9, wherein the predetermined threshold value is at least 10 times a Lower Limit of Detection of the assay.

16. The method of claim 9, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

17. The method of claim 16, wherein the tauopathy is Alzheimer's disease.

18. The method of claim 16, wherein the tauopathy is progressive supranuclear palsy.

19. A method of detecting amyloidogenic disease in a subject, the method comprising:
obtaining a plasma sample from the subject;
detecting an amount of p217+tau peptides in the plasma sample using an assay, wherein the assay uses a capture antibody directed against a p217+tau epitope to bind the capture antibody to the p217+tau peptides in the plasma sample to form antibody-peptide complexes and a detection antibody to bind the detection antibody to the antibody-peptide complexes; and
determining the subject as having amyloidogenic disease or is at risk of developing amyloidogenic disease when the amount of the p217+tau peptides is above a predetermined threshold value, wherein the predetermined threshold value is above a Lower Limit of Quantification (LLOQ) of the assay.

20. The method of claim 19, wherein the assay does not concentrate the p217+tau peptides from the plasma sample by immunoprecipitation before measuring the amount of the p217+tau peptides present.

21. The method of claim 19, wherein the plasma sample is crude plasma.

22. The method of claim 19, wherein the LLOQ corresponds to a 15-25% coefficient of variation (CV) of the assay.

23. The method of claim 22, wherein the LLOQ corresponds to a 20% CV of the assay.

24. The method of claim 19, wherein the predetermined threshold value is at least 3 times of the LLOQ.

25. The method of claim 19, wherein the predetermined threshold value is at least 10 times a Lower Limit of Quantification of the assay.

26. The method of claim 19, wherein the amyloidogenic disease is Alzheimer's disease.

27. A method for detecting or predicting tauopathy in a subject, the method comprising:
detecting an amount of p217+tau peptides in a plasma sample by contacting the plasma sample with a capture antibody directed against a p217+tau epitope to bind the capture antibody to the p217+tau peptides in the plasma sample to form antibody-peptide complexes, and separately contacting the antibody-peptide complexes with a detection antibody to bind the detection antibody to the antibody-peptide complexes;
generating tau data corresponding to the amount of the p217+tau peptides detected;
obtaining biomarker data corresponding to at least one biomarker detected from the subject, wherein the biomarker is selected from a group comprising NFL, adiponectin and leptin; and
comparing the tau data and further biomarker data to a set of reference data using a machine learning module to determine or predict whether the subject has tauopathy or is at risk of developing tauopathy.

28. The method of claim 27, wherein the machine learning module comprises at least one of a support vector machine module, a random forest module, a logistic regression module, and a gradient boosting module.

29. The method of claim 27, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

30. The method of claim 27, wherein the tauopathy is Alzheimer's disease.

31. The method of claim 27, wherein the tauopathy is progressive supranuclear palsy.
